(19) United States
(12) Patent Application Publication
(10) Pub. No.: US 2025/0318798 A1
(43) Pub. Date: Oct. 16, 2025

(54) MEDICAL SYSTEM AND STORING MEDIUM

(71) Applicant: GE Precision Healthcare LLC, Waukesha, WI (US)

(72) Inventors: Koichi Hoshino, Hino (JP); Yasuhiro Imai, Hino (JP)

(21) Appl. No.: 19/175,616

(22) Filed: Apr. 10, 2025

(30) Foreign Application Priority Data
Apr. 10, 2024 (JP) 2024-063670

(51) Int. Cl. A61B 6/58; A61B 6/03

(52) U.S. Cl. CPC A61B 6/585; A61B 6/032

(57) ABSTRACT

Systems and methods to notify a user that an X-ray generating device may be deteriorating due to aging before the image quality of a CT image deteriorates are described. A medical system may include an X-ray generating device, a detector for detecting X-rays irradiated from the X-ray generating device, and one or more processors that execute instructions to calculate a characteristic value of the X-rays irradiated from the X-ray generating device based on data on the X-rays detected by the detector, every time a prescribed calibration scan is executed, and determine whether to output an alert based on change in the characteristic value over time.

### CROSS-REFERENCE TO RELATED APPLICATIONS

This application claims priority to Japanese Application No. 2024-063670, filed on Apr. 10, 2024 the disclosure of which is incorporated herein by reference in its entirety.

### TECHNICAL FIELD

The present invention relates to a medical system for irradiating X-rays, and to a storing medium on which an instruction for controlling the medical system is recorded.

### BACKGROUND ART

A CT system is known as a medical system that noninvasively images a subject. CT systems can acquire tomographic images of a subject in a short scanning time and therefore are widely used in hospitals and other medical facilities.

The CT system applies a prescribed voltage to a cathode-anode tube of an X-ray tube to generate X-rays. The generated X-rays penetrate the subject and are detected by a detector. The CT system reconstructs a CT image of the subject based on data detected by the detector.

### SUMMARY OF THE INVENTION

Single energy CT (SECT) is a well-known imaging technique for CT systems. SECT is a method for obtaining a CT image of a subject by applying a prescribed voltage (e.g., 120 kV) to a cathode-anode tube of an X-ray tube to generate X-rays. However, in SECT, CT values may be close even for different substances, and identification of different substances may be difficult.

Therefore, DECT (Dual Energy CT) technology is being researched and developed. DECT is a technology that can use X-rays in different energy regions to discriminate between substances, and can acquire images that are useful for diagnosis in clinical settings, and thus is beginning to come into widespread use. In the DECT technology, a kV switching technology is known that switches the tube voltage of the X-ray tube between a low tube voltage and a high tube voltage.

On the other hand, the accuracy of substance discrimination using the kV switching method of DECT decreases with age-related deterioration of the X-ray generating device (e.g., X-ray tube, generator, and the like). Therefore, in order to prevent a decrease in the accuracy of substance discrimination, it is important for a user of a CT system to discover the age-related deterioration of the X-ray generating device and the like as soon as possible. However, in many cases, no effort is made to detect deterioration of the X-ray generating device, and the user of the CT system becomes aware of deterioration of the X-ray generating device when the deterioration appears as noise or artifacts in a CT image. Therefore, there is a demand for a technology that can inform the user that the X-ray generating device may be deteriorating due to aging before the image quality of a CT image deteriorates (before noise or artifacts appear in the CT image).

A first aspect of the present invention is a medical system including an X-ray generating device, a detector for detecting X-rays irradiated from the X-ray generating device, and one or a plurality of processors. The processes are operative to calculate a characteristic value of the X-rays irradiated from the X-ray generating device based on data on the X-rays detected by the detector, every time a prescribed calibration scan is executed, and determine whether to output an alert based on change in the characteristic value over time.

A second aspect of the present invention is a non-transitory computer-readable storage medium included in or in communication with a medical system. The medical system includes an X-ray generating device, a detector for detecting X-rays irradiated from the X-ray generating device, and one or a plurality of processors. Instructions stored in the storing medium, when executed by the one or more processors, causes the one or more processors to calculate a characteristic value of the X-rays irradiated from the X-ray generating device based on data on the X-rays detected by the detector, every time a prescribed calibration scan is executed, and determine whether to output an alert based on change in the characteristic value over time.

In the present invention, a characteristic value of X-rays irradiated from the X-ray generating device is calculated based on data including information on the X-rays detected by the detector, and a determination is made as to whether to output an alert based on change in the characteristic value over time. Therefore, by outputting an alert when there is a large change over time in the characteristic value, a user can be notified that there are signs of deterioration in the X-ray generating device before the image quality of a CT image deteriorates.

### BRIEF DESCRIPTION OF THE DRAWINGS

FIG. 1 is a block diagram of a CT system 10;

FIG. 2 is a diagram depicting a flow executed on each medical examination day;

FIG. 3 is an explanatory diagram of calibration;

FIG. 4 is an explanatory diagram of calibration data acquired on medical examination day 1;

FIG. 5 is an enlarged view of absorption coefficient data g₁₁;

FIG. 6 is an enlarged view of absorption coefficient data h₁₁;

FIG. 7 is a diagram depicting absorption coefficient data g₂₁ for a first reference substance obtained on medical examination day 2;

FIG. 8 is a diagram depicting absorption coefficient data h₂₁ for a second reference substance obtained on medical examination day 2;

FIG. 9 is a diagram depicting an example of a flow of step ST20;

FIG. 10 is an explanatory diagram of the flow of FIG. 9;

FIG. 11 is an explanatory diagram of steps ST201 and ST202 executed for channel 2;

FIG. 12 is an explanatory diagram of steps ST201 and ST202 executed for channel j;

FIG. 13 is an explanatory diagram of steps ST201 and ST202 executed for channel n;

FIG. 14 is an explanatory diagram when step ST20 is executed for absorption coefficient data of the second reference substance;

FIG. 15 is a diagram depicting absorption coefficient data g₁₁ to g₃₁ for the first reference substance obtained on medical examination days 1 to 3;

FIG. 16 is a diagram depicting absorption coefficient data h₁₁ to h₃₁ for the second reference substance obtained on medical examination days 1 to 3;

FIG. 17 is an explanatory diagram of step ST20;

FIG. 18 is an explanatory diagram when steps ST201 to ST204 are executed for absorption coefficient data of the second reference substance;

FIG. 19 is a diagram depicting absorption coefficient data gᵢ₁ for a first reference substance obtained on medical examination day i;

FIG. 20 is a diagram depicting absorption coefficient data hᵢ₁ for a second reference substance obtained on medical examination day i;

FIG. 21 is an explanatory diagram of step ST20;

FIG. 22 is an explanatory diagram of a flow after an operation of the CT system is restarted;

FIG. 23 is a diagram depicting absorption coefficient data gₚ₁ for a first reference substance obtained on medical examination day p;

FIG. 24 is a diagram depicting absorption coefficient data hₚ₁ for a second reference substance obtained on medical examination day p;

FIG. 25 is a diagram depicting absorption coefficient data g_{q1} for a first reference substance obtained on medical examination day q;

FIG. 26 is a diagram depicting absorption coefficient data h_{q1} for a second reference substance obtained on medical examination day q;

FIG. 27 is an explanatory diagram of step ST20;

FIG. 28 is an explanatory diagram when steps ST201 to ST204 are executed for absorption coefficient data of the second reference substance;

FIG. 29 is a diagram depicting an example of a flow of step ST20 when a detector with a plurality of rows is used;

FIG. 30 is an explanatory diagram of a flow of step ST20 when updating the reference absorption coefficient for each medical examination day;

FIG. 31 is a diagram depicting the absorption coefficient data g₁₁ to gᵢ₁ of the first reference substance acquired on medical examination days 1 to i;

FIG. 32 is an explanatory diagram of a flow performed in another excample;

FIG. 33 is an explanatory diagram of an X-ray spectrum;

FIG. 34 is a diagram schematically depicting an X-ray spectrum SL2 corresponding to low kV generated on medical examination day 2, and an X-ray spectrum SH2 corresponding to high kV generated on medical examination day 2;

FIG. 35 is a diagram depicting an example of a flow of step ST60;

FIG. 36 is an explanatory diagram of the flow of FIG. 35;

FIG. 37 is an explanatory diagram of steps ST604 to ST606;

FIG. 38 is a diagram schematically depicting an X-ray spectrum SLi corresponding to low kV generated on medical examination day i, and an X-ray spectrum SHi corresponding to high kV generated on medical examination day i;

FIG. 39 is an explanatory diagram of step ST60;

FIG. 40 is an explanatory diagram of a flow after an operation of the CT system is restarted;

FIG. 41 is an explanatory diagram of a flow of step ST60 when calculating an energy reference value for each medical examination day; and

FIG. 42 is a diagram depicting X-ray spectrums SL1 to SLᵢ corresponding to low kV acquired on medical examination days 1 to i.

### DETAILED DESCRIPTION OF THE DRAWINGS

Embodiments for carrying out the invention will be described below, but the present invention is not limited to the following embodiments.

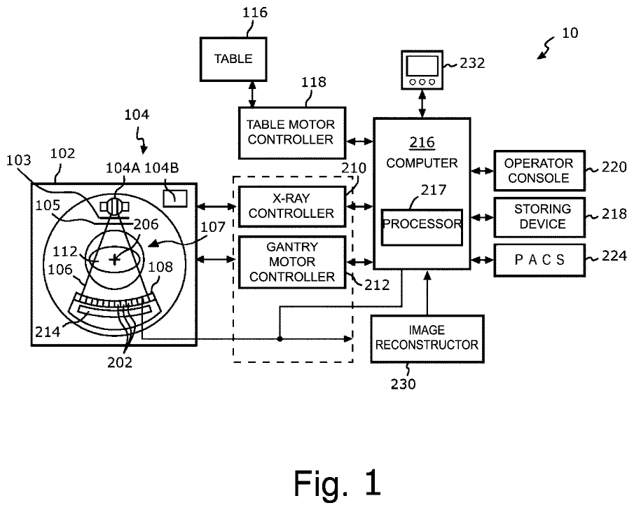

FIG. 1 is a block diagram of a CT system 10. The CT system 10 includes a gantry 102 and a table 116. The gantry 102 includes a bore 107. A subject 112 is transported through the bore 107 and then the subject 112 is scanned. An X-ray generating device 104, a filter part 103, a pre-collimator 105, a detector 108, and the like are attached to the gantry 102. The X-ray generating device 104 includes an X-ray tube 104A and a generator 104B. The generator 104B supplies power to X-ray tube 104A. The X-ray tube 104A outputs X-rays when a prescribed voltage is applied to a cathode-anode tube. The X-ray tube 104 is configured to be rotatable on a path centered on a rotation axis 206 within the XY plane. Herein, the Z direction represents the body-axis direction, the Y direction represents the vertical direction (the height direction of the table 116), and the X direction represents the direction perpendicular to the Z and Y directions. In the present example embodiment, the X-ray tube 104A supports a kV switching scheme in which the tube voltage applied to the X-ray tube can be alternately switched between a first tube voltage and a second tube voltage. Note that in the present embodiment, the CT system 10 includes one X-ray tube 104A, but may include two X-ray tubes 104A.

The filter part 103 includes, for example, a flat plate filter and/or a bow-tie filter. The pre-collimator 105 is a member that narrows the X-ray irradiation range such that X-rays are not irradiated in unwanted regions. The detector 108 includes a plurality of detector elements 202. The plurality of detector elements 202 detect an X-ray beam 106 that is irradiated from the X-ray tube 104A and passes through the subject 112, such as a patient or the like. Therefore, the X-ray detector 108 can acquire projection data for each view.

The projection data detected by the X-ray detector 108 is acquired by a DAS 214. The DAS 214 executes prescribed processing, including sampling, digital conversion, and the like, on the acquired projection data. The processed projection data is transmitted to a computer 216. The computer 216 stores the data from the DAS 214 in a storing device 218. The storing device 218 includes one or more storing medium that records a program, instruction, and the like to be executed by the processor. The storing medium may be, for example, one or more non-transitory computer-readable storing medium. The storing device 218 may include, for example, hard disk drives, floppy disk drives, compact disc read/write (CD-R/W) drives, digital versatile disk (DVD) drives, flash drives, and/or solid state recording drives.

The computer 216 includes one or a plurality of processors 217. The computer 216 uses one or a plurality of processors to output commands and parameters to the DAS 214, X-ray controller 210, and/or gantry motor controller 212, to control system operations such as data acquisition and/or processing. Furthermore, the computer 216 uses one or a plurality of processors to execute various processes such as signal processing, data processing, image processing, and the like in each step of the flow (to be described later). Note that in FIG. 1, one or a plurality of the processors 217 are included in the computer 216, but one or a plurality of the processors 217 may be provided so as to be distributed between the computer 216 and another component (e.g., X-ray controller 210, gantry motor controller 212, table controller 118, or the like).

An operator console 220 is linked to the computer 216. An operator can enter prescribed operator inputs related to an operation of the CT system 10 into the computer 216 by operating the operator console 220. The computer 216 receives an operator input, including a command and/or scan parameter, via the operator console 220 and controls system operation based on the operator input. The operator console 220 can include a keyboard (not depicted) or touch screen for the operator to specify a command and/or scan parameter.

The X-ray controller 210 controls the X-ray generating device 104 based on an instruction from the computer 216. Furthermore, the gantry motor controller 212 controls a gantry motor to rotate a component, such as the X-ray tube 104A, detector 108, or the like, based on an instruction from the computer 216.

FIG. 1 depicts only one operator console 220, but two or more operator consoles may be linked to the computer 216. Furthermore, the CT system 10 may also allow a plurality of remotely located displays, printers, workstations, and/or similar devices to be linked via, for example, a wired and/or wireless network. In one embodiment, for example, the CT system 10 may include a Picture Archiving and Communication System (PACS) 224, or may be linked to the PACS 224. In a typical implementation, a PACS 224 may be linked to a remote system such as a radiology department information system, hospital information system, and/or internal or external network (not depicted) or the like.

The computer 216 provides an instruction to a table motor controller 118 to control the table 116. The table motor controller 118 can control the table motor so as to move the table 116 based on the instructions received. For example, the table motor controller 118 can move the table 116 such that the subject 112 is positioned appropriately for imaging.

As mentioned above, the DAS 214 samples and digitally converts the projection data acquired by the detector elements 202. The image reconstructor 230 then reconstructs the image using the sampled and digitally converted data. The image reconstructor 230 includes one or a plurality of processors, which can execute image reconstruction processing. In FIG. 1, the image reconstructor 230 is depicted as a separate component from the computer 216, but the image reconstructor 230 may form a part of the computer 216. Furthermore, the computer 216 may also perform one or a plurality of functions of the image reconstructor 230. Furthermore, the image reconstructor 230 may be positioned away from the CT system 10 and operatively connected to the CT system 10 using a wired or wireless network.

The image reconstructor 230 can store the reconstructed image in the storing device 218. The image reconstructor 230 may also transmit the reconstructed image to the computer 216. The computer 216 can transmit the reconstructed image and/or patient information to a display device 232 communicatively linked to the computer 216 and/or image reconstructor 230.

The various methods and processes described in the present specification can be recorded as executable instructions on a non-transitory computer-readable storing medium included in or in communication with the CT system 10. The executable instructions may be recorded on a single storing medium or distributed across a plurality of storing media. One or more processors provided in the CT system 10 executes the various methods, steps, and processes described in the present specifications in accordance with the instructions recorded on a storing medium.

The CT system 10 is configured as described above. The CT system of the present embodiment is compatible with the kV switching method of DECT (Dual Energy CT) and can discriminate substances. However, the accuracy of substance discrimination using the kV switching method of DECT decreases with age-related deterioration of the X-ray generating device. Therefore, in order to prevent a decrease in the accuracy of substance discrimination, it is important for a user of the CT system to discover age-related deterioration of the X-ray generating device as soon as possible. However, currently there has been no attempt to detect deterioration of X-ray generating devices. Therefore, users of CT systems become aware of deterioration of the X-ray generating device by visually recognizing a decrease in the image quality of a CT image. For this reason, there is a demand for a technology that notifies a user that the X-ray generating device may be deteriorating due to aging before the image quality of a CT image deteriorates.

Therefore, the present inventors conducted extensive research and conceived of a method for notifying a user of a CT system that an X-ray generating device may be deteriorating due to aging before the user visually recognizes a deterioration in the image quality of a CT image. This method will be described below.

Before describing the present embodiment in detail, features of the present embodiment will be summarized as follows. In the present embodiment, a calibration scan is periodically executed. When the calibration scan is executed, the processor calculates a characteristic value of X-rays irradiated from the X-ray generating device 104 based on data of the X-rays detected by the detector 108. Furthermore, the processor determines whether to output an alert based on change in the characteristic value over time. Therefore, by outputting an alert when there is a large change over time in the characteristic value, a user can be notified that there are signs of deterioration in the X-ray generating device before the image quality of a CT image deteriorates.

Furthermore, it is recommended that the calibration scan is periodically executed (e.g., every morning) in order to obtain stable CT images. Therefore, if the calibration scan is periodically executed as before, an alert is output as necessary to notify the user. This allows the user to be notified that the X-ray generating device is showing signs of deterioration before the image quality of a CT image deteriorates, without having to execute an additional scan apart from the calibration scan.

A method for determining whether to output an alert based on a change over time in the characteristic value of X-rays in the CT system of the present embodiment will be described below. Note that two characteristic values of X-rays are considered below, namely, the absorption coefficient of a first reference substance and the absorption coefficient of a second reference substance.

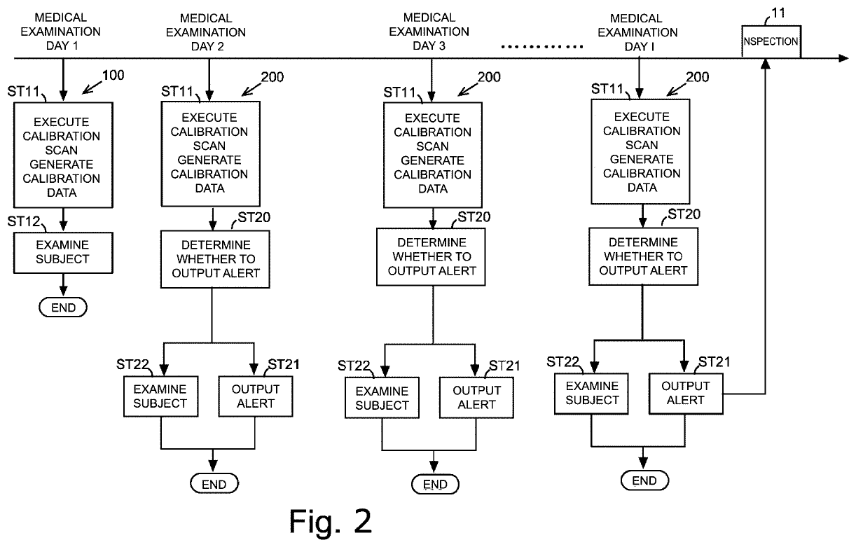

FIG. 2 is a diagram depicting a flow executed on each medical examination day. For convenience of description, this method will be described below using an example in which a calibration scan is executed once per medical examination day, but the frequency of execution of the calibration scan is not limited to once a day. For example, the calibration scan may be executed a plurality of times a day (e.g., in the morning and afternoon) or there may be periodically set days on which the calibration scan is not executed.

First, medical examination day 1 will be described. On medical examination day 1, a flow 100 is executed. The flow 100 will be described below. In step ST11, a calibration scan is executed using kV switching in which the tube voltage applied to the X-ray tube 104A is alternately switched between a first tube voltage (low kV) and a second tube voltage (high kV).

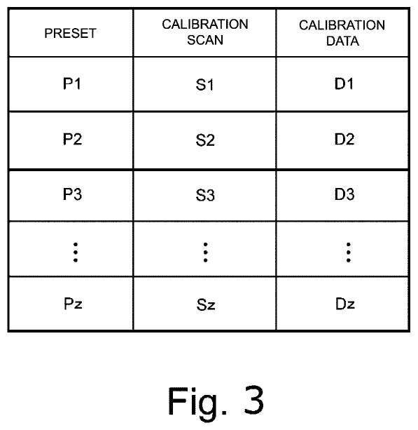

FIG. 3 is an explanatory diagram of calibration. In the calibration of the kV switching method, for example, z-number of combinations P1 to Pz of values of three parameters (rotation speed, cone angle, and tube current) are prepared. Furthermore, z-number of combinations P1 to Pz are preset and stored in the CT system, and calibration scans S1 to Sz are executed for each combination. By executing the calibration scans, X-rays are detected by the detector 108. The processor generates calibration data based on the X-ray data detected by the detector 108. FIG. 2 depicts an example in which z-number of pieces of calibration data D1 to Dz are generated for z-number of presets P1 to Pz. Each piece of calibration data includes various coefficients, such as an absorption coefficient, a correction coefficient for beam hardening, and the like. However, herein, only calibration data representing an absorption coefficient related to the description of the present embodiment is considered as the calibration data.

Note that when analyzing the absorption coefficient, it is not necessary to analyze the absorption coefficients of all the calibration data D1 to Dz, but it is sufficient to analyze the change in the absorption coefficient over time of the calibration data obtained in a specific calibration scan of the calibration scans S1 to Sz. Therefore, the absorption coefficient is described below with a focus only on the calibration data D1 obtained by the calibration scan S1.

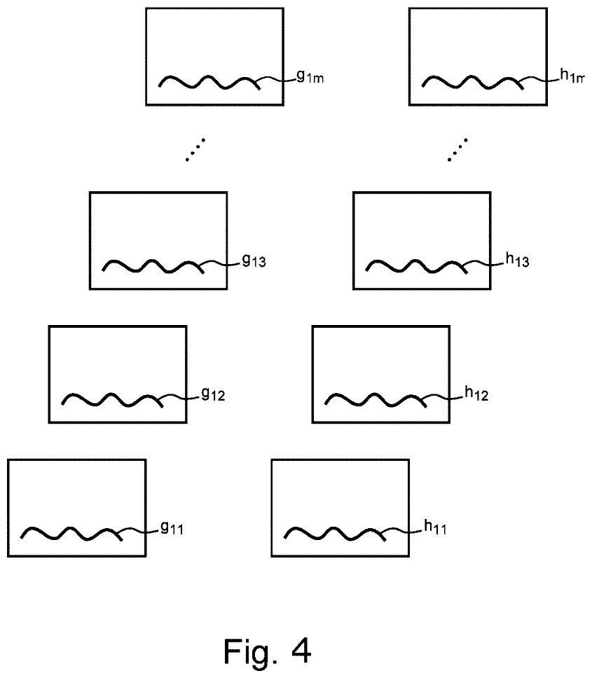

The absorption coefficients include an absorption coefficient of a first reference substance and an absorption coefficient of a second reference substance. Therefore, by executing a calibration scan, the absorption coefficient of the first reference substance and the absorption coefficient of the second reference substance are calculated. The two reference substances may be, for example, a combination of water and iodine, or a combination of water and calcium. FIG. 4 is an explanatory diagram of the calibration data acquired on medical examination day 1.

FIG. 4 depicts an outline of absorption coefficient data g₁₁ to g₁ₘ representing the absorption coefficients of the first reference substance and absorption coefficient data h₁₁ to h₁ₘ representing the absorption coefficients of the second reference substance of the calibration data acquired on medical examination day 1.

The absorption coefficient data g₁₁ represents the absorption coefficient of the first reference substance in each channel of a first row of the detector, and the absorption coefficient data g₁₂ to g₁ₘ represent the absorption coefficients of the first reference substance in each channel of a second row to an m-th row of the detector, respectively. Furthermore, the absorption coefficient data h₁₁ represents the absorption coefficient of the second reference substance in each channel of a first row of the detector, and the absorption coefficient data h₁₂ to him represent the absorption coefficients of the second reference substance in each channel of a second row to an m-th row of the detector, respectively. For example, when m=64, in other words, when the detector has a multi-row structure with 64 rows, 64 pieces of absorption coefficient data g₁₁ to g_{1,64} are generated for the first reference substance, and 64 pieces of absorption coefficient data h₁₁ to h_{1,64} are generated for the second reference substance. Note that in order to simplify the description of the absorption coefficient data, a case where m=1, in other words, the detector has a single-row structure, is considered below. Therefore, for the first reference substance, only the absorption coefficient data g₁₁ is considered, and for the second reference substance, only the absorption coefficient data h₁₁ is considered.

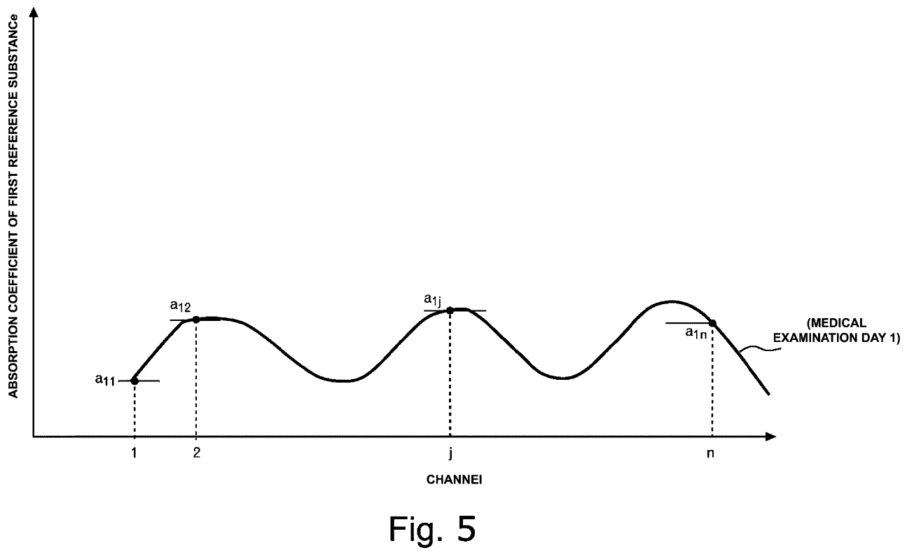

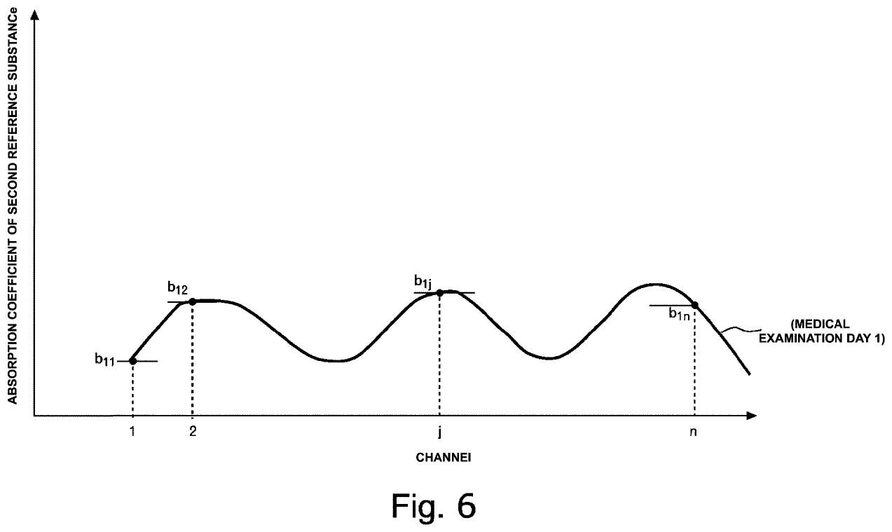

FIG. 5 is an enlarged view of the absorption coefficient data g₁₁, and FIG. 6 is an enlarged view of the absorption coefficient data h₁₁. Note that waveforms of the absorption coefficient data below are indicated for the purpose of describing the embodiment, and may differ from actual waveforms.

FIG. 5 depicts only the absorption coefficients of channel 1, channel 2, channel j, and channel n as representatives of the absorption coefficients of the first reference substance in channels 1 to n of the detector. In channel 1, the absorption coefficient is denoted as “a₁₁”, in channel 2, the absorption coefficient is denoted as “a₁₂”, in channel j, the absorption coefficient is denoted as “a₁ⱼ”, and in channel n, the absorption coefficient is denoted as “a₁ₙ”.

FIG. 6 depicts only the absorption coefficients of channel 1, channel 2, channel j, and channel n as representatives of the absorption coefficients of the second reference substance in channels 1 to n of the detector. In channel 1, the absorption coefficient is denoted as “b₁₁”, in channel 2, the absorption coefficient is denoted as “b₁₂”, in channel j, the absorption coefficient is denoted as “b₁ⱼ”, and in channel n, the absorption coefficient is denoted as “b₁ₙ”.

In the present embodiment, the absorption coefficients a₁₁ to a₁ₙ of the first reference substance calculated on medical examination day 1 are stored as first reference absorption coefficients serving as criteria for determining whether or not to output an alert. Furthermore, the absorption coefficients b₁₁ to b₁ₙ of the second reference substance calculated on medical examination day 1 are stored as second reference absorption coefficients serving as criteria for determining whether or not to output an alert.

After step ST11 is executed, the process proceeds to step ST12, where examination of a subject is executed in accordance with the examination schedule for medical examination day 1. Next, medical examination day 2 will be described. On medical examination day 2, a flow 200 is executed. First, in step ST11, a calibration scan is executed. The processor generates calibration data (absorption coefficient data or the like) based on data obtained from the calibration scan.

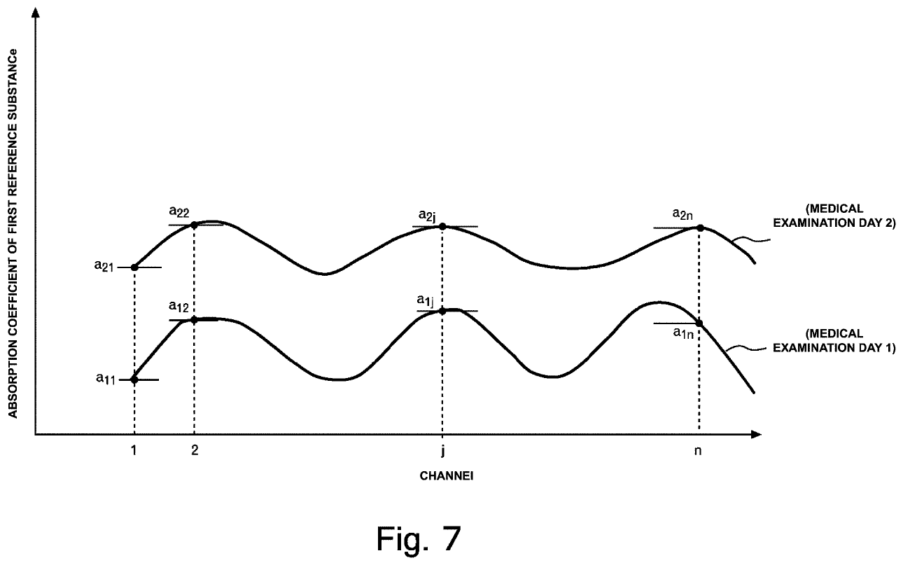

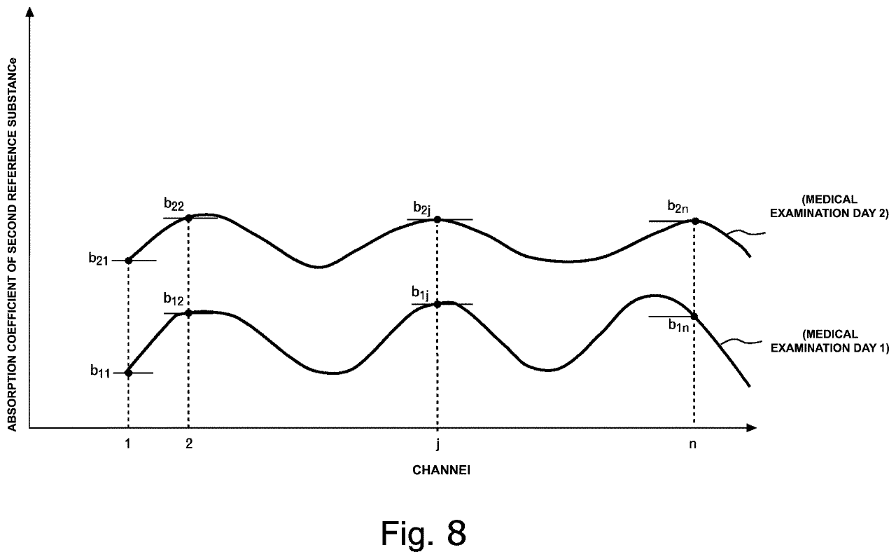

FIG. 7 is a diagram depicting absorption coefficient data g₂₁ of the first reference substance obtained on medical examination day 2, and FIG. 8 is a diagram depicting absorption coefficient data h₂₁ of the second reference substance obtained on medical examination day 2.

Note that in addition to the absorption coefficient data g₂₁ obtained on medical examination day 2, FIG. 7 also depicts the absorption coefficient data g₁₁ obtained on medical examination day 1. Furthermore, in addition to the absorption coefficient data h₂₁ obtained on medical examination day 2, FIG. 8 also depicts the absorption coefficient data h₁₁ obtained on medical examination day 1. After step ST11 is executed, the process proceeds to step ST20. In step ST20, whether or not to output an alert is determined based on change in the absorption coefficient over time.

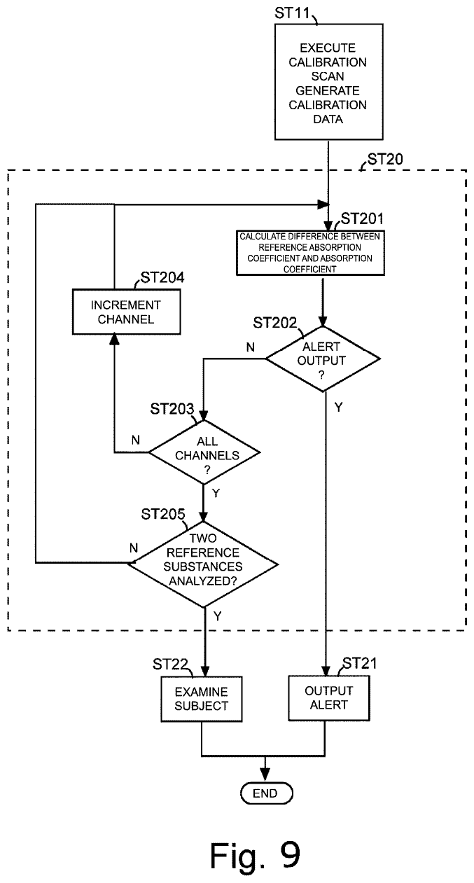

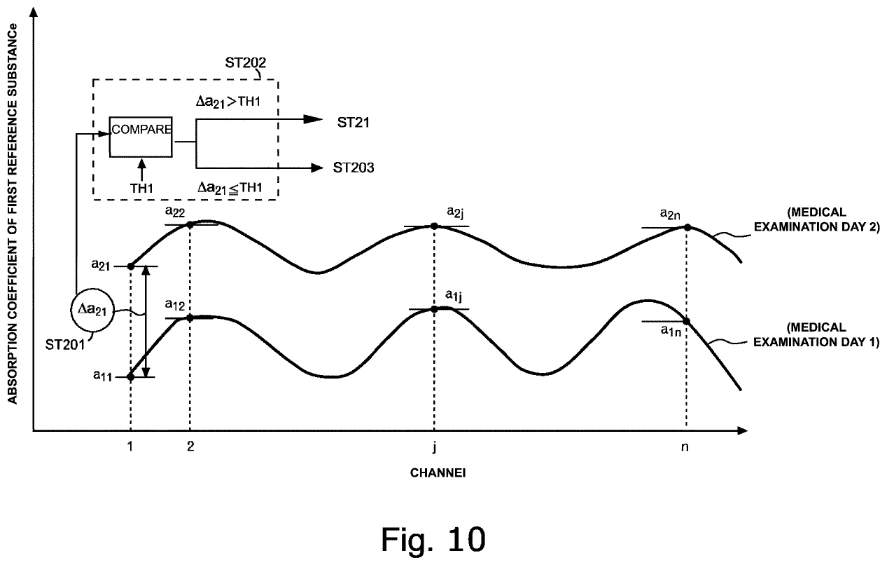

FIG. 9 is a diagram depicting an example of a flow of step ST20, and FIG. 10 is an explanatory diagram of the flow of FIG. 9. FIG. 10 depicts the absorption coefficient data g₁₁ and g₂₁ of the first reference substance depicted in FIG. 7. In step ST201, the processor calculates the difference between the reference absorption coefficient (absorption coefficient of medical examination day 1) and the absorption coefficient of medical examination day 2. Specifically, the processor calculates a difference Δa₂₁ between a reference absorption coefficient a₁₁ of medical examination day 1 and an absorption coefficient a₂₁ of medical examination day 2 in channel 1. Once the absorption coefficient difference Δa₂₁ has been calculated, the process proceeds to step ST202.

In step ST202, the processor compares Δa₂₁ with a threshold value TH1, and determines whether or not to output an alert based on the comparison result thereof. For example, when Δa₂₁ exceeds the threshold value TH1 (Δa₂₁>TH1), the processor determines that the change in the absorption coefficient over time is large and therefore determines to output an alert. In this case, the process proceeds to step ST21, where the processor causes the display unit to output an alert.

On the other hand, if the difference Δa₂₁ in the absorption coefficients does not exceed the threshold value TH1 (Δa₂₁≤TH1), the change in the absorption coefficient over time is small, and therefore, the processor determines not to output an alert. In this case, the process proceeds to step ST203.

In step ST203, the processor determines whether or not steps ST201 and ST202 have been executed for all channels. Herein, steps ST201 and ST202 have been executed for channel 1, but have not yet been executed for the other channels 2 to n. Therefore, the process proceeds to step ST204, where the processor increments the channel from channel 1 to channel 2. Once the channel is incremented, the process returns to step ST201.

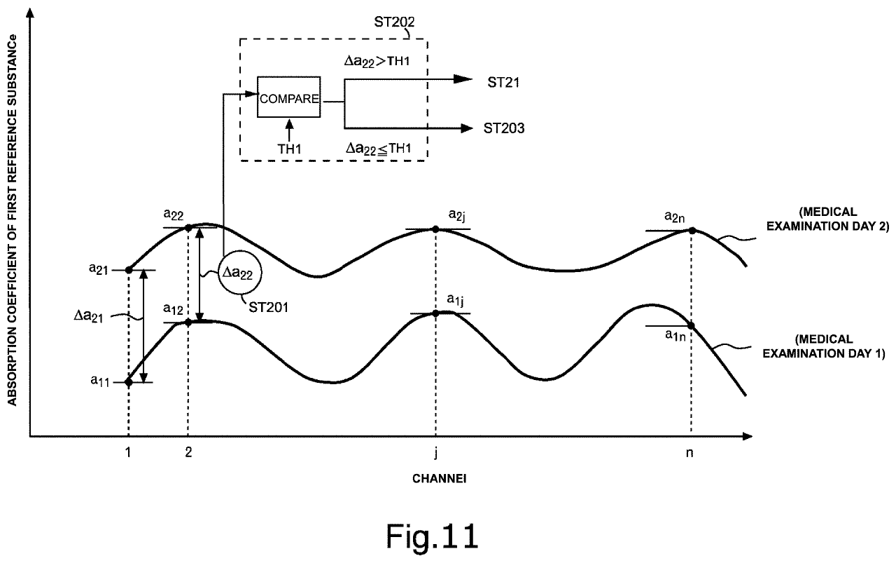

FIG. 11 is an explanatory diagram of steps ST201 and ST202 executed for channel 2. In step ST201, the processor calculates a difference Δa₂₂ between an absorption coefficient a₁₂ of medical examination day 1 and an absorption coefficient a₂₂ of medical examination day 2 in channel 2. Once the absorption coefficient difference Δa₂₂ has been calculated, the process proceeds to step ST202.

In step ST202, the processor compares Δa₂₂ with a threshold value TH1, and determines whether or not to output an alert based on the comparison result thereof. For example, when Δa₂₂ exceeds the threshold value TH1 (Δa₂₂>TH1), the processor determines that the change in the absorption coefficient over time is large and therefore determines to output an alert. In this case, the process proceeds to step ST21, where the processor causes the display unit to output an alert.

On the other hand, if the difference Δa₂₂ in the absorption coefficients does not exceed the threshold value TH1 (Δa₂₂≤TH1), the change in the absorption coefficient over time is small, and therefore, the processor determines not to output an alert. In this case, the process proceeds to step ST203.

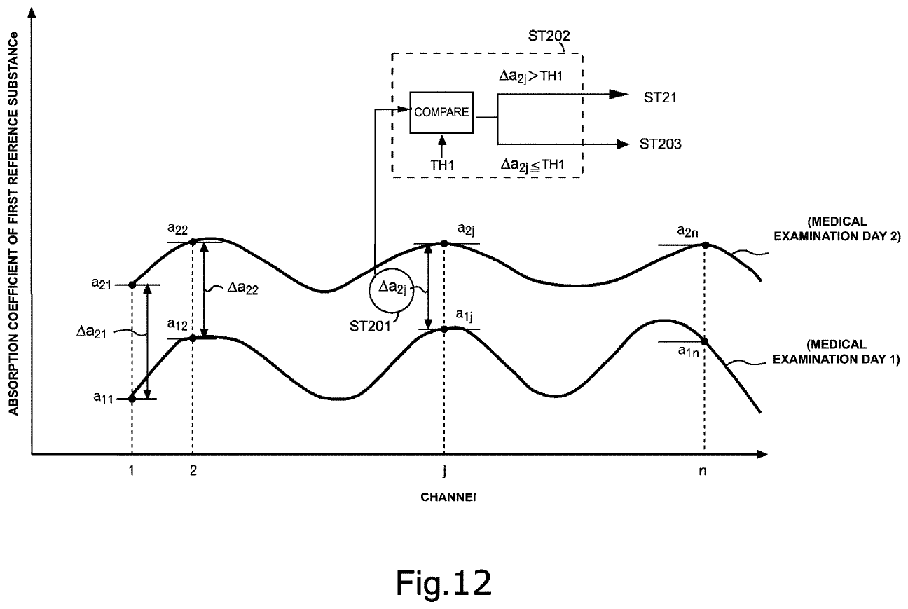

In step ST203, the processor determines whether or not steps ST201 and ST202 have been executed for all channels. Herein, steps ST201 and ST202 have been executed for channels 1 and 2, but have not yet been executed for the other channels 3 to n. Therefore, the process proceeds to step ST204, where the processor increments the channel from channel 2 to channel 3. The process then returns to step ST201. Similarly hereinafter, every time the channel is incremented in step ST204, the processor returns to step ST201 and repeatedly executes a loop of steps ST201 to ST204 (see FIG. 12).

FIG. 12 is an explanatory diagram of steps ST201 and ST202 executed for channel j. In step ST201, the processor calculates a difference Δa₂ⱼ between an absorption coefficient a₁ⱼ of medical examination day 1 and an absorption coefficient a₂ⱼ of medical examination day 2 in channel j. Once the absorption coefficient difference Δa₂ⱼ has been calculated, the process proceeds to step ST202.

In step ST202, the processor compares Δa₂ⱼ with a threshold value TH1, and determines whether or not to output an alert based on the comparison result thereof. For example, when Δa₂ⱼ exceeds the threshold value TH1 (Δa₂ⱼ>TH1), the processor determines that the change in the absorption coefficient over time is large and therefore determines to output an alert. In this case, the process proceeds to step ST21, where the processor causes the display unit to output an alert.

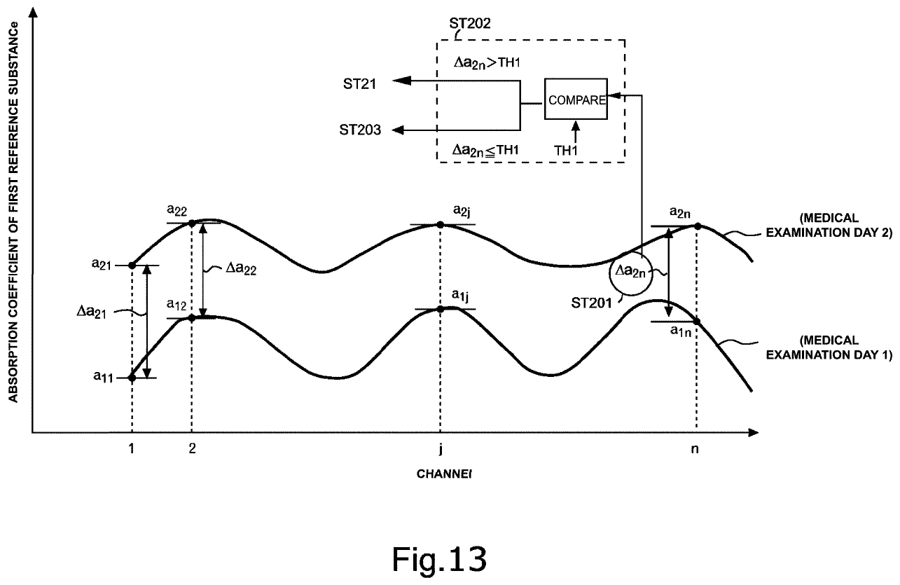

On the other hand, if the difference Δa₂ⱼ in the absorption coefficients does not exceed the threshold value TH1 (Δa₂ⱼ≤TH1), the change in the absorption coefficient over time is small, and therefore, the processor determines not to output an alert. In this case, the process proceeds to step ST203. Similarly, every time the channel is incremented in step ST204, the processor returns to step ST201 and repeatedly executes a loop of steps ST201 to ST204 (see FIG. 13).

FIG. 13 is an explanatory diagram of steps ST201 and ST202 executed for channel n. In step ST201, the processor calculates a difference Δa₂ₙ between an absorption coefficient a₁ₙ of medical examination day 1 and an absorption coefficient a₂ₙ of medical examination day 2 in channel n. Once the absorption coefficient difference Δa₂ₙ has been calculated, the process proceeds to step ST202.

In step ST202, the processor compares Δa₂ₙ with a threshold value TH1, and determines whether or not to output an alert based on the comparison result thereof. For example, when Δa₂ₙ exceeds the threshold value TH1 (Δa₂ₙ>TH1), the processor determines that the change in the absorption coefficient over time is large and therefore determines to output an alert. In this case, the process proceeds to step ST21, where the processor causes the display unit to output an alert.

On the other hand, if the difference Δa₂ₙ in the absorption coefficients does not exceed the threshold value TH1 (Δa₂ₙ≤TH1), the change in the absorption coefficient over time is small, and therefore, the processor determines not to output an alert. In this case, the process proceeds to step ST203.

In step ST203, the processor determines whether or not steps ST201 to ST204 have been executed for all channels. Herein, steps ST201 to ST204 are executed for all channels 1 to n. Therefore, the process proceeds to step ST205.

In step ST205, the processor determines whether or not analysis of the absorption coefficients of both the first and second reference substances is completed. Herein, the absorption coefficient of the first reference substance was analyzed, but the absorption coefficient of the second reference substance has not been analyzed. Therefore, the process returns to step ST201.

Returning to step ST201, the processor executes the processes of steps ST201 to ST204 for the absorption coefficient data h₁₁ and h₂₁ (see FIG. 8) of the second reference substance as described for the first reference substance.

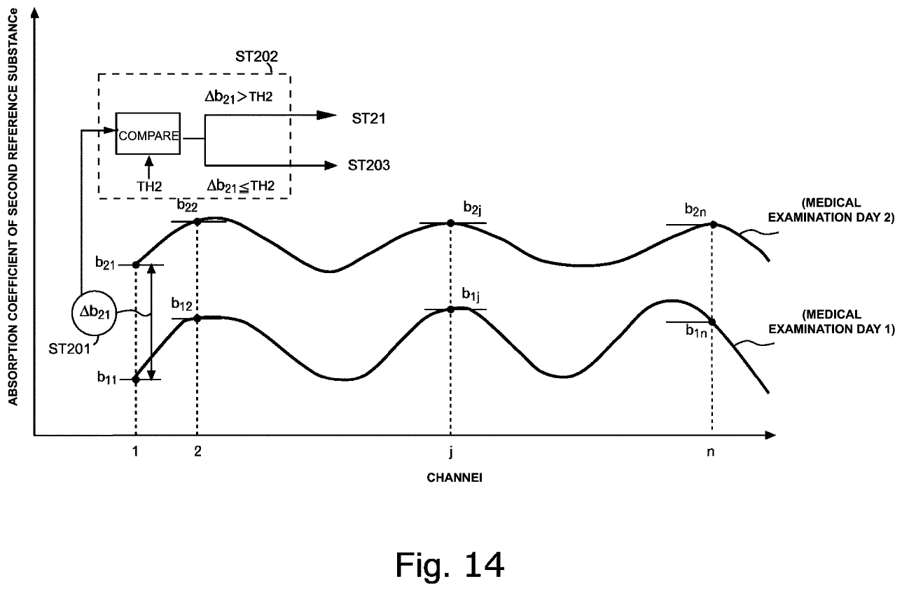

FIG. 14 is an explanatory diagram when step ST20 is executed for absorption coefficient data of the second reference substance. FIG. 14 depicts the absorption coefficient data h₁₁ and h₂₁ of the second reference substance depicted in FIG. 8.

In step ST201, the processor calculates a difference Δb₂₁ between a reference absorption coefficient b₁₁ of medical examination day 1 and an absorption coefficient b₂₁ of medical examination day 2 in channel 1. Once the absorption coefficient difference Δb₂₁ has been calculated, the process proceeds to step ST202.

In step ST202, the processor compares Δb₂₁ with a threshold value TH2, and determines whether or not to output an alert based on the comparison result thereof. For example, when Δb₂₁ exceeds the threshold value TH2 (Δb₂₁>TH2), the processor determines that the change in the absorption coefficient over time is large and therefore determines to output an alert. In this case, the process proceeds to step ST21, where the processor causes the display unit to output an alert.

On the other hand, if the difference Δb₂₁ in the absorption coefficients does not exceed the threshold value TH2 (Δb₂₁≤TH2), the change in the absorption coefficient over time is small, and therefore, the processor determines not to output an alert. In this case, the process proceeds to step ST203.

In step ST203, the processor determines whether or not steps ST201 and ST202 have been executed for all channels. Herein, steps ST201 and ST202 have been executed for channel 1, but have not yet been executed for the other channels 2 to n. Therefore, the process proceeds to step ST204, where the processor increments the channel from channel 1 to channel 2. Once the channel is incremented, the process returns to step ST201. Similarly hereinafter, the loop of steps ST201 to ST204 is executed. Furthermore, while the processes of steps ST201 to ST204 are executed for channels 1 to n of the absorption coefficient data h₁₁ and h₂₁, if the difference in absorption coefficient in a certain channel exceeds the threshold value TH2, it is determined that an alert is to be output. On the other hand, if the processor has executed steps ST201 to ST204 for all channels of the absorption coefficient data h₁₁ and h₁₂ of the second reference substance and the difference in absorption coefficient is equal to or less than the threshold value TH2 in any channel, the processor proceeds to step ST205.

In step ST205, the processor determines whether or not analysis of the absorption coefficients of both the first and second reference substances is completed. Herein, the analysis of the absorption coefficients of both the first and second reference substances has been completed. Therefore, the processor determines that output of an alert is not necessary, and ends step ST20. In this case, the process proceeds to step ST22, where examination of the subject is executed in accordance with the examination schedule for medical examination day 2. In the present embodiment, an alert was not output on medical examination day 2. Therefore, after step ST20 ends, examination of the subject is executed. Returning to FIG. 2, the description is continued.

Next, medical examination day 3 will be described. On medical examination day 3, the flow 200 is executed in the same manner as on medical examination day 2. First, in step ST11, a calibration scan is executed. The processor generates calibration data (absorption coefficient data or the like) based on data obtained from the calibration scan.

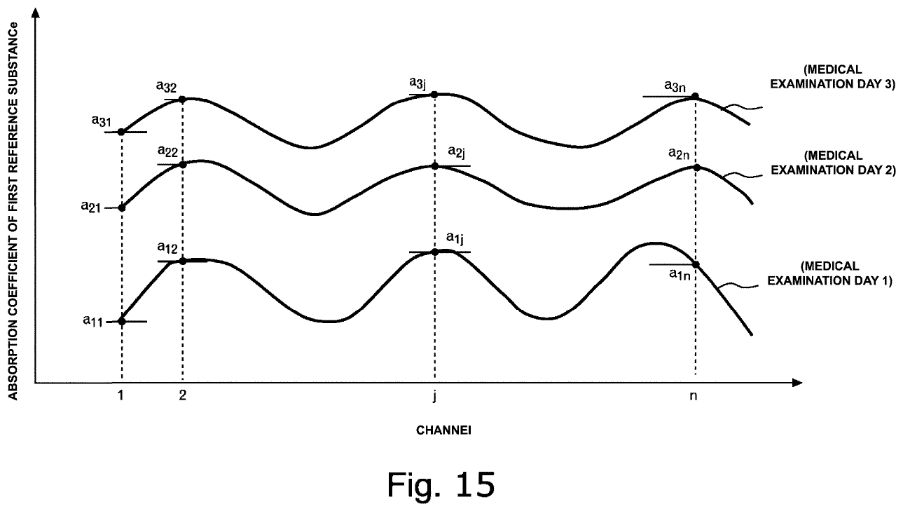

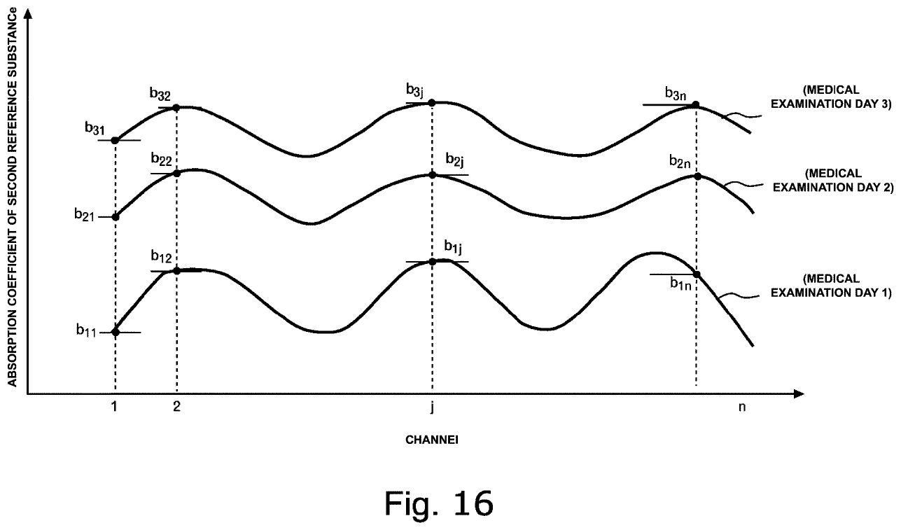

FIG. 15 depicts absorption coefficient data g₁₁ to g₃₁ of the first reference substance obtained on medical examination days 1 to 3, and FIG. 16 depicts absorption coefficient data h₁₁ to h₃₁ of the second reference substance obtained on medical examination days 1 to 3.

After step ST11 is executed, the process proceeds to step ST20. In step ST20, whether or not to output an alert is determined based on change in the absorption coefficient over time.

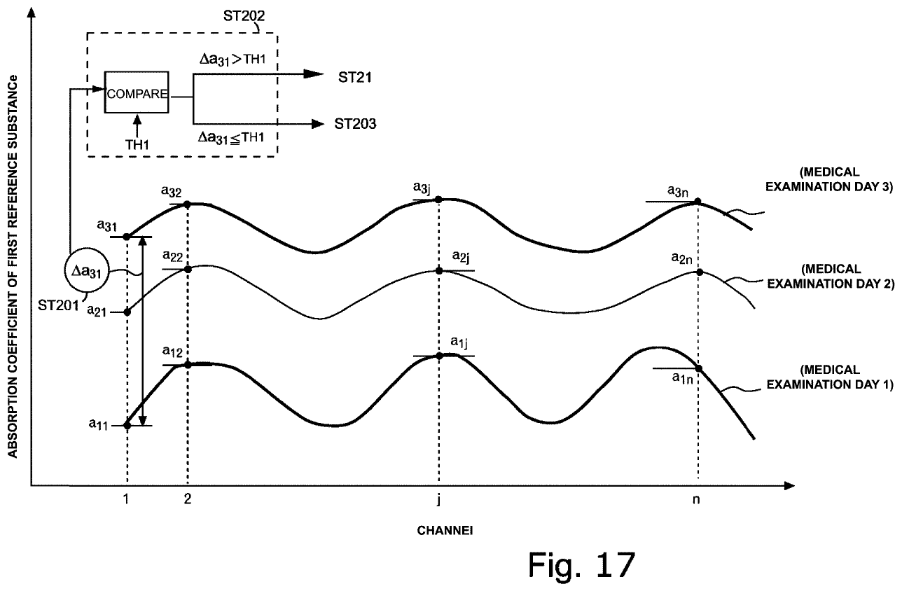

FIG. 17 is an explanatory diagram of step ST20. In step ST201, the processor calculates a difference Δa₃₁ between the reference absorption coefficient a₁₁ of medical examination day 1 and an absorption coefficient a₃₁ of medical examination day 2 in channel 1. Once the absorption coefficient difference Δa₃₁ has been calculated, the process proceeds to step ST202.

In step ST202, the processor compares Δa₃₁ with a threshold value TH1, and determines whether or not to output an alert based on the comparison result thereof. For example, when Δa₃₁ exceeds the threshold value TH1 (Δa₃₁>TH1), the processor determines that the change in the absorption coefficient over time is large and therefore determines to output an alert. In this case, the process proceeds to step ST21, where the processor causes the display unit to output an alert.

On the other hand, if the difference Δa₃₁ in the absorption coefficients does not exceed the threshold value TH1 (Δa₃₁≤TH1), the change in the absorption coefficient over time is small, and therefore, the processor determines not to output an alert. In this case, the process proceeds to step ST203.

In step ST203, the processor determines whether or not steps ST201 and ST202 have been executed for all channels. Herein, steps ST201 and ST202 have been executed for channel 1, but have not yet been executed for the other channels 2 to n. Therefore, the process proceeds to step ST204, where the processor increments the channel from channel 1 to channel 2. Once the channel is incremented, the process returns to step ST201.

Similarly hereinafter, the loop of steps ST201 to ST204 is executed. Furthermore, while the processes of steps ST201 to ST204 are executed for channels 1 to n of the absorption coefficient data g₁₁ and g₃₁, if the difference in absorption coefficient in a certain channel exceeds the threshold value TH1, it is determined that an alert is to be output.

On the other hand, if the processor has executed steps ST201 to ST204 for all channels of the absorption coefficient data g₁₁ and g₃₁ of the first reference substance and the difference in absorption coefficient is equal to or less than the threshold value TH1 in any channel, the processor proceeds to step ST205. In step ST205, the processor determines whether or not analysis of the absorption coefficients of both the first and second reference substances is completed. Herein, the absorption coefficient of the first reference substance was completed, but the absorption coefficient of the second reference substance has not been analyzed. Therefore, the process returns to step ST201. Returning to step ST201, the processor executes the processes of steps ST201 to ST204 for the absorption coefficient of the second reference substance.

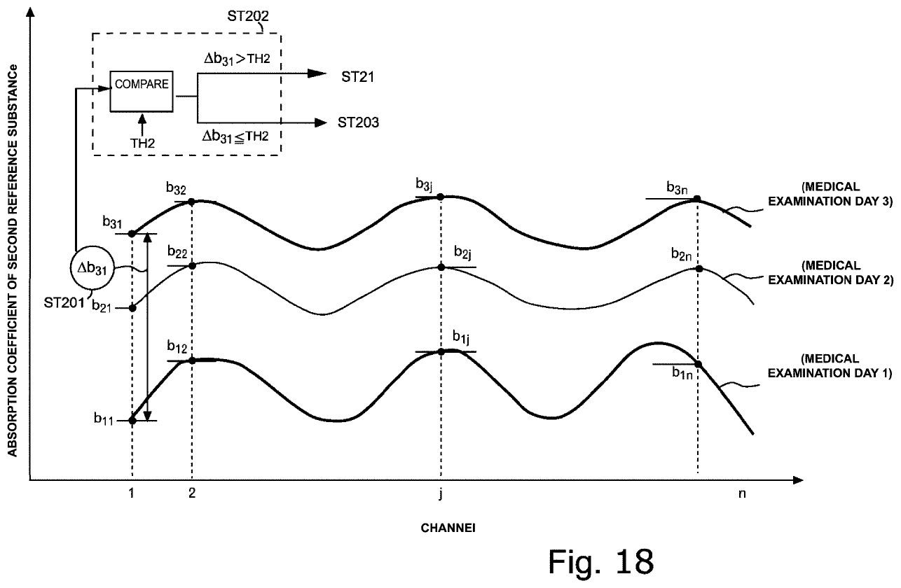

FIG. 18 is an explanatory diagram when steps ST201 to ST204 are executed for the absorption coefficient data of the second reference substance. In step ST201, the processor calculates a difference Δb₂₁ between the absorption coefficient b₁₁ of medical examination day 1 and an absorption coefficient b₂₁ of medical examination day 2 in channel 1. Once the absorption coefficient difference Δb₃₁ has been calculated, the process proceeds to step ST202.

In step ST202, the processor compares Δb₃₁ with a threshold value TH2, and determines whether or not to output an alert based on the comparison result thereof. For example, when Δb₃₁ exceeds the threshold value TH2 (Δb₃₁>TH2), the processor determines that the change in the absorption coefficient over time is large and therefore determines to output an alert. In this case, the process proceeds to step ST21, where the processor causes the display unit to output an alert. On the other hand, if the difference Δb₃₁ in the absorption coefficients does not exceed the threshold value TH2 (Δb₃₁≤TH2), the change in the absorption coefficient over time is small, and therefore, the processor determines not to output an alert. In this case, the process proceeds to step ST203.

In step ST203, the processor determines whether or not steps ST201 and ST202 have been executed for all channels. Herein, steps ST201 and ST202 have been executed for channel 1, but have not yet been executed for the other channels 2 to n. Therefore, the process proceeds to step ST204, where the processor increments the channel from channel 1 to channel 2. Once the channel is incremented, the process returns to step ST201.

Similarly hereinafter, the loop of steps ST201 to ST204 is executed. Furthermore, while the processes of steps ST201 to ST204 are executed for channels 1 to n of the absorption coefficient data h₁₁ and h₃₁, if the difference in absorption coefficient in a certain channel exceeds the threshold value TH2, it is determined that an alert is to be output.

On the other hand, if the processor has executed steps ST201 to ST204 for all channels of the absorption coefficient data h₁₁ and h₁₃ of the second reference substance and the difference in absorption coefficient is equal to or less than the threshold value TH2 in any channel, the processor proceeds to step ST205.

In step ST205, the processor determines whether or not analysis of the absorption coefficients of both the first and second reference substances is completed. Herein, the analysis of the absorption coefficients of both the first reference substance and second reference substance has been completed. Therefore, the processor determines that output of an alert is not necessary, and ends step ST20. In this case, the process proceeds to step ST22, where examination of the subject is executed in accordance with the examination schedule for medical examination day 3. In the present embodiment, an alert was not output on medical examination day 3. Therefore, after step ST20 ends, examination of the subject is executed.

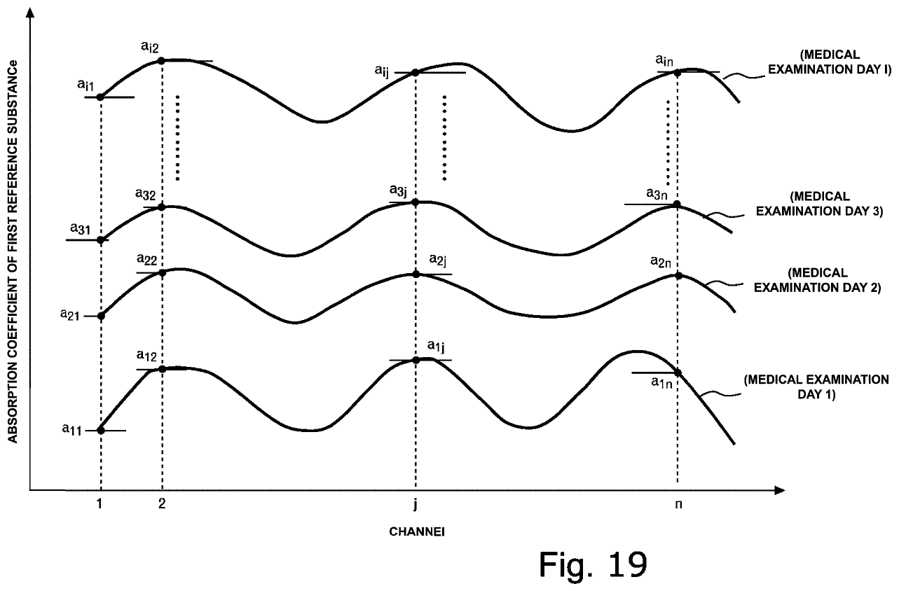

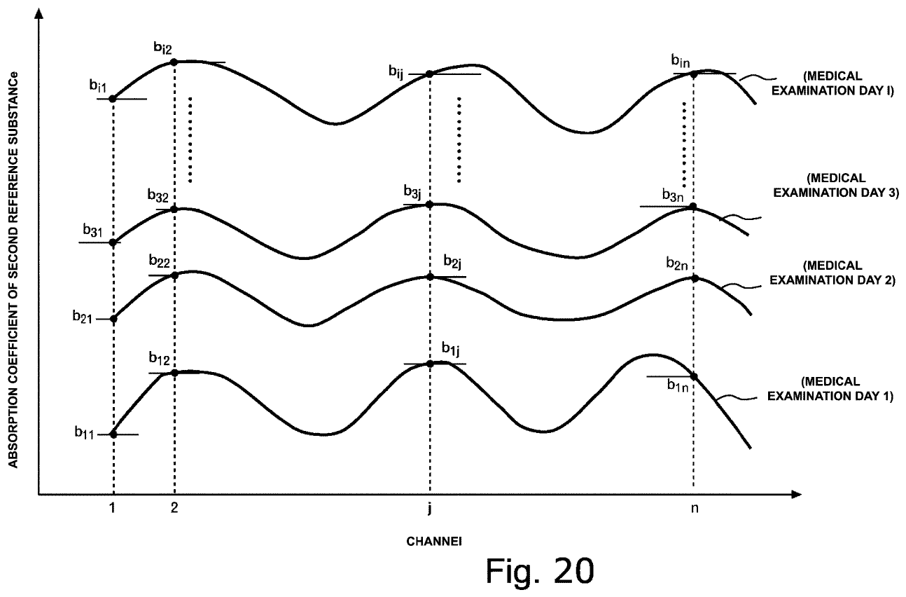

Next, medical examination day i will be described. On medical examination day i, the flow 200 is executed in the same manner as on medical examination day 2. In step ST11, a calibration scan is executed. The processor generates calibration data (absorption coefficient data or the like) based on data obtained from the calibration scan. FIG. 19 depicts absorption coefficient data gᵢ₁ of the first reference substance obtained on medical examination day i, and FIG. 20 depicts absorption coefficient data hᵢ₁ of the second reference substance obtained on medical examination day i. After step ST11 is executed, the process proceeds to step ST20. In step ST20, whether or not to output an alert is determined based on change in the absorption coefficient over time.

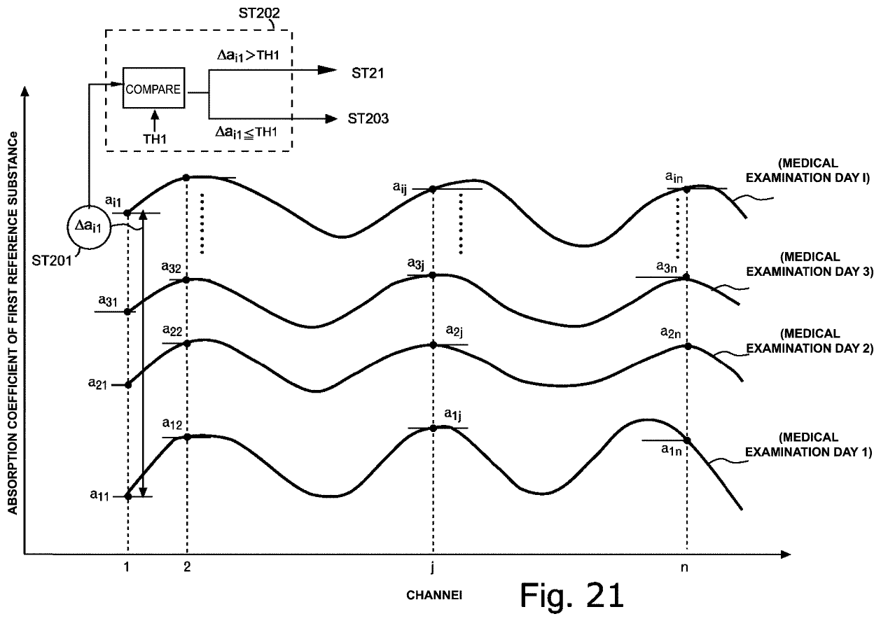

FIG. 21 is an explanatory diagram of step ST20. FIG. 21 depicts absorption coefficient data g₁₁ to gᵢ₁ acquired on medical examination days 1 to i. In step ST201, the processor calculates a difference Δaᵢ₁ between the absorption coefficient a₁₁ of medical examination day 1 and an absorption coefficient aᵢ₁ of medical examination day i in channel 1. Once the absorption coefficient difference Δaᵢ₁ has been calculated, the process proceeds to step ST202.

In step ST202, the processor compares Δaᵢ₁ with a threshold value TH1, and determines whether or not to output an alert based on the comparison result thereof. For example, when Δaᵢ₁ exceeds the threshold value TH1 (Δaᵢ₁>TH1), the processor determines that the change in the absorption coefficient over time is large and therefore determines to output an alert. In this case, the process proceeds to step ST21, where the processor causes the display unit to output an alert.

On the other hand, if the difference Δaᵢ₁ in the absorption coefficients does not exceed the threshold value TH1 (Δaᵢ₁≤TH1), the change in the absorption coefficient over time is small, and therefore, the processor determines not to output an alert. In this case, the process proceeds to step ST203. Herein, the difference Δaᵢ₁ of the absorption coefficient calculated in channel 1 satisfies Δaᵢ₁>TH1. Therefore, an alert is output. When the alert is output, the user can, for example, request a service to carry out an inspection 11 (see FIG. 2). Furthermore, when an alert is output, the CT system may send data representing a time change of the absorption coefficient to a back office, and the data representing the time change of the absorption coefficient may be analyzed in the back office.

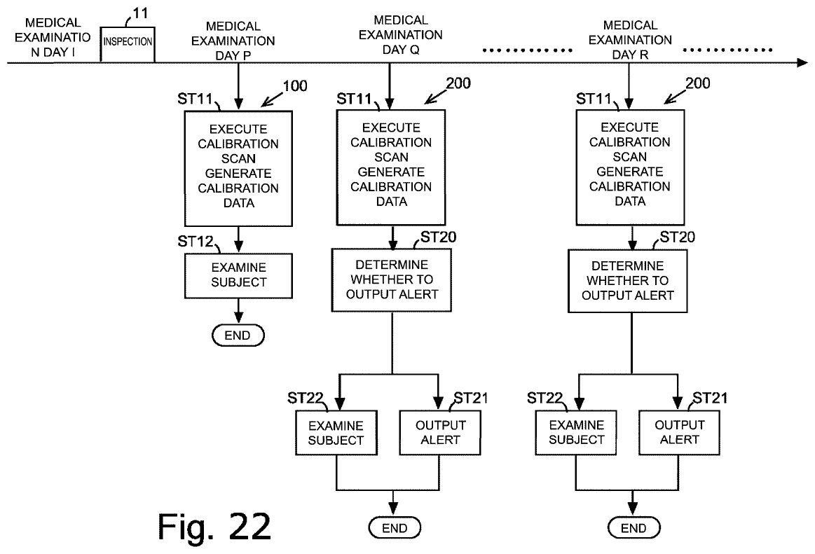

As a result of the service inspection 11, if the X-ray generating device is acknowledged to have malfunctioned or failed, the X-ray generating device is repaired or replaced. After the X-ray generating device is repaired or replaced, an operation of the CT system is checked, and if the CT system is confirmed to be operating normally, the operation of the CT system is resumed. FIG. 22 is an explanatory diagram of a flow after the operation of the CT system is restarted. Herein, the inspection or the like is completed on medical examination day i, and operation of the CT system is resumed on a subsequent medical examination day p. Medical examination day p will be described below.

On medical examination day p, the same flow 100 as medical examination day 1 is executed. Therefore, in step ST11, a calibration scan is executed. The processor generates calibration data (absorption coefficient data or the like) based on data obtained from the calibration scan.

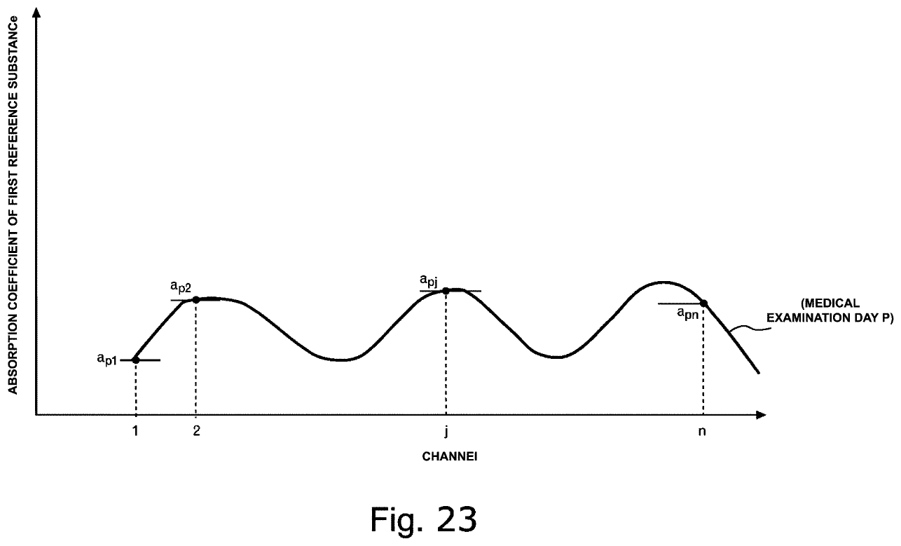

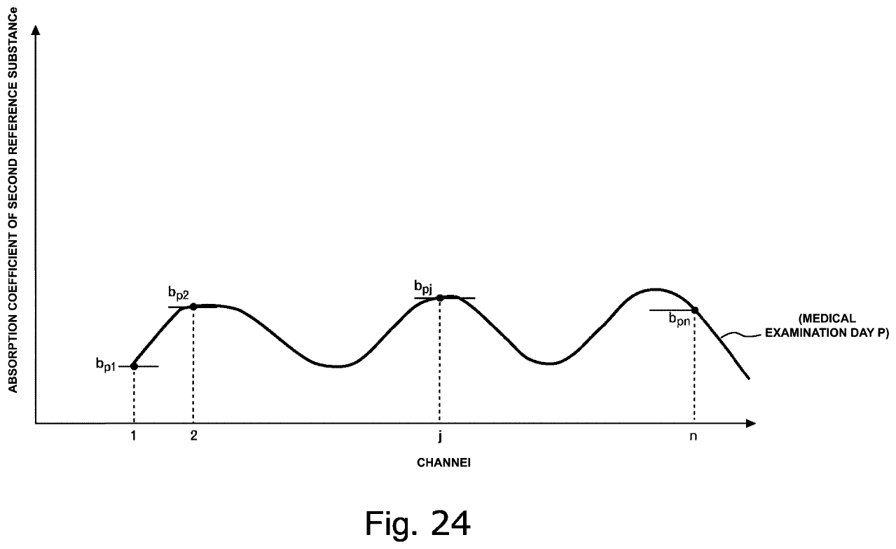

FIG. 23 is a diagram depicting absorption coefficient data gₚ₁ of the first reference substance obtained on medical examination day p, and FIG. 24 is a diagram depicting absorption coefficient data hₚ₁ of the second reference substance obtained on medical examination day p.

The absorption coefficient data gₚ₁ represents the absorption coefficient of the first reference substance in each channel of the first row of the detector. Furthermore, the absorption coefficient data hₚ₁ represents the absorption coefficient of the second reference substance in each channel of the first row of the detector. FIGS. 23 and 24 depict only the absorption coefficients of channel 1, channel 2, channel j, and channel n as representatives of the absorption coefficients of the first reference substance in channels 1 to n of the detector.

On medical examination days 1 to i, the absorption coefficients a₁₁ to a₁ₙ of the first reference substance acquired on medical examination day 1 were used as reference values for determining the amount of change over time in the absorption coefficients (e.g., absorption coefficients a₂₁, a₃₁, aᵢ₁) of the first reference substance acquired on medical examination days 2 to i. However, the X-ray generating device is inspected on medical examination day i, and the operation of the CT system is resumed on the subsequent medical examination day p. Therefore, the absorption coefficients a₁₁ to a₁ₙ of medical examination day 1 acquired before the inspection of the X-ray generating device cannot be used as reference values for determining the amount of change over time in the absorption coefficient of the first reference substance. Therefore, the processor stores absorption coefficients aₚ₁ to aₚₙ acquired on medical examination day p as new first reference values for determining the amount of change over time in the absorption coefficient of the first reference substance.

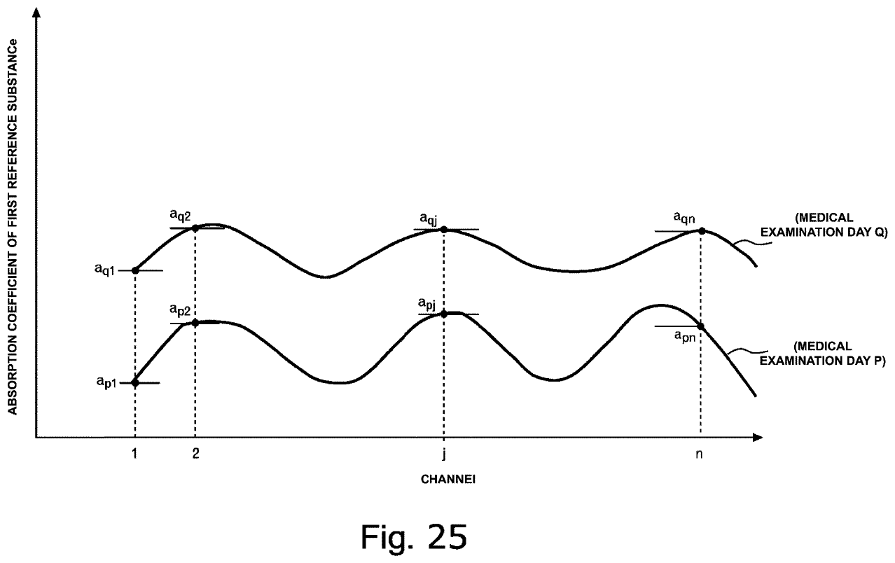

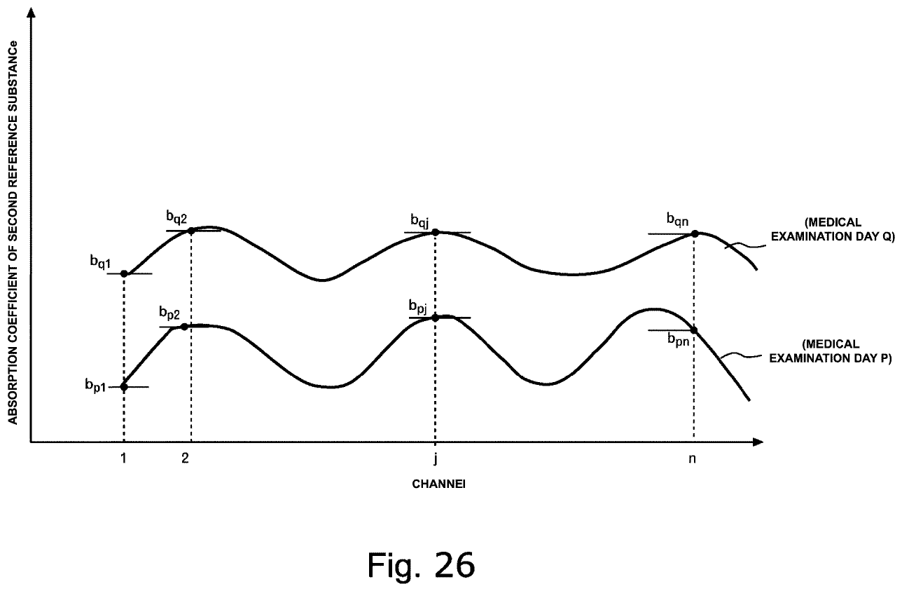

Similarly, the processor stores absorption coefficients bₚ₁ to bₚₙ acquired on medical examination day p as new second reference values for determining the amount of change over time in the absorption coefficient of the second reference substance. After step ST11 is executed, the process proceeds to step ST12, where examination of the subject is executed. Next, medical examination day q will be described. On medical examination day q, the flow 200 is executed in the same manner as on medical examination day 2. Therefore, in step ST11, a calibration scan is executed. The processor generates calibration data (absorption coefficient data or the like) based on data obtained from the calibration scan. FIG. 25 depicts absorption coefficient data g_{q1} of the first reference substance obtained on medical examination day q, and FIG. 26 depicts absorption coefficient data h_{q1} of the second reference substance obtained on medical examination day q. After step ST11 is executed, the process proceeds to step ST20. In step ST20, whether or not to output an alert is determined based on change in the absorption coefficient over time.

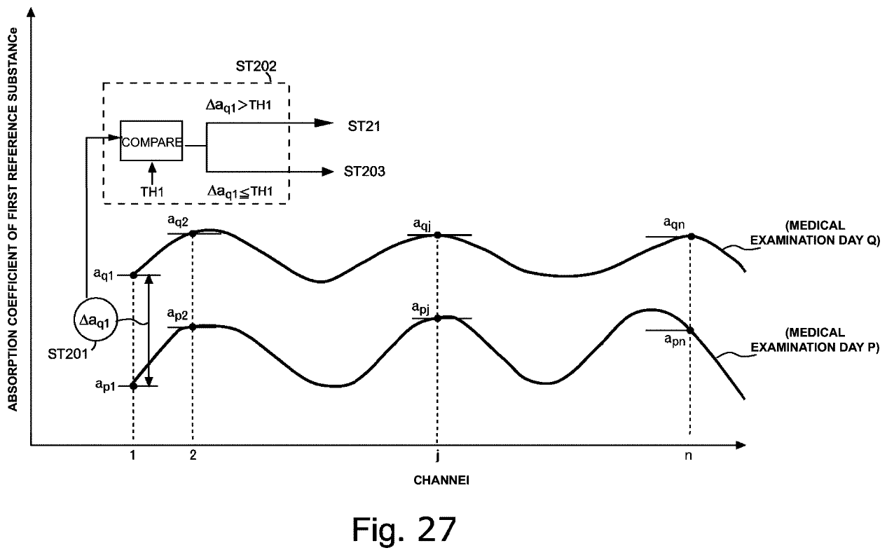

FIG. 27 is an explanatory diagram of step ST20. In step ST201, the processor calculates a difference Δaₚ₁ between the absorption coefficient a_{q1} of medical examination day p and an absorption coefficient aᵢ₁ of medical examination day q in channel 1. Once the absorption coefficient difference Δa_{q1} has been calculated, the process proceeds to step ST202.

In step ST202, the processor compares Δa_{q1} with a threshold value TH1, and determines whether or not to output an alert based on the comparison result thereof. For example, when Δa_{q1} exceeds the threshold value TH1 (Δa_{q1}>TH1), the processor determines that the change in the absorption coefficient over time is large and therefore determines to output an alert. In this case, the process proceeds to step ST21, where the processor causes the display unit to output an alert.

On the other hand, if the difference Δa_{q1} in the absorption coefficients does not exceed the threshold value TH1 (Δa_{q1}≤TH1), the change in the absorption coefficient over time is small, and therefore, the processor determines not to output an alert. In this case, the process proceeds to step ST203.

In step ST203, the processor determines whether or not steps ST201 and ST202 have been executed for all channels. Herein, steps ST201 and ST202 have been executed for channel 1, but have not yet been executed for the other channels 2 to n. Therefore, the process proceeds to step ST204, where the processor increments the channel from channel 1 to channel 2. Once the channel is incremented, the process returns to step ST201.

Similarly hereinafter, the loop of steps ST201 to ST204 is executed. Furthermore, while the processes of steps ST201 to ST204 are executed for channels 1 to n of the absorption coefficient data gₚ₁ and g_{q1}, if the difference in absorption coefficient in a certain channel exceeds the threshold value TH1, it is determined that an alert is to be output.

On the other hand, if the processor has executed steps ST201 to ST204 for all channels of the absorption coefficient data g₁₁ and g₃₁ of the first reference substance and the difference in absorption coefficient is equal to or less than the threshold value TH1 in any channel, the processor proceeds to step ST205. In step ST205, the processor determines whether or not analysis of the absorption coefficients of both the first and second reference substances is completed. Herein, the absorption coefficient of the first reference substance was analyzed, but the absorption coefficient of the second reference substance has not been analyzed. Therefore, the process returns to step ST201.

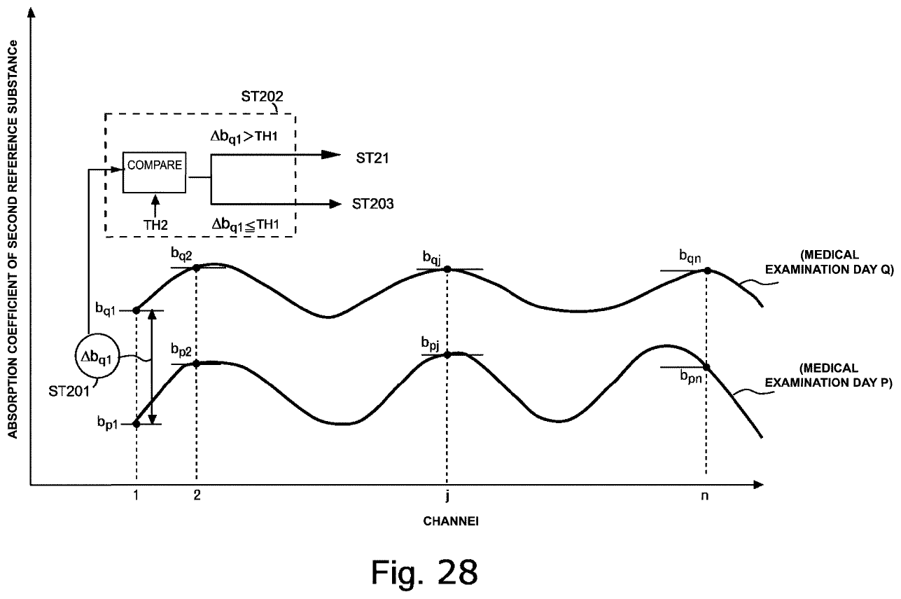

Returning to step ST201, the processor executes the processes of steps ST201 to ST204 for the absorption coefficient of the second reference substance. FIG. 28 is an explanatory diagram when steps ST201 to ST204 are executed for the absorption coefficient data of the second reference substance. In step ST201, the processor calculates a difference Δbₚ₁ between the absorption coefficient bₚ₁ of medical examination day p and an absorption coefficient b_{q1} of medical examination day q in channel 1. Once the absorption coefficient difference Δb_{q1} has been calculated, the process proceeds to step ST202.

In step ST202, the processor compares Δb_{q1} with a threshold value TH2, and determines whether or not to output an alert based on the comparison result thereof. For example, when Δb_{q1} exceeds the threshold value TH2 (Δb_{q1}>TH2), the processor determines that the change in the absorption coefficient over time is large and therefore determines to output an alert. In this case, the process proceeds to step ST21, where the processor causes the display unit to output an alert.

On the other hand, if the difference Δb_{q1} in the absorption coefficients does not exceed the threshold value TH2 (Δb_{q1}≤TH2), the change in the absorption coefficient over time is small, and therefore, the processor determines not to output an alert. In this case, the process proceeds to step ST203.

In step ST203, the processor determines whether or not steps ST201 and ST202 have been executed for all channels. Herein, steps ST201 and ST202 have been executed for channel 1, but have not yet been executed for the other channels 2 to n. Therefore, the process proceeds to step ST204, where the processor increments the channel from channel 1 to channel 2. Once the channel is incremented, the process returns to step ST201.

Similarly, hereinafter, the loop of steps ST201 to ST204 is executed. Furthermore, while the processes of steps ST201 to ST204 are executed for channels 1 to n of the absorption coefficient data h₁₁ and h_{q1}, if the difference in absorption coefficient in a certain channel exceeds the threshold value TH2, it is determined that an alert is to be output.

On the other hand, if the processor has executed steps ST201 to ST204 for all channels of the absorption coefficient data h₁₁ and h₁₃ of the second reference substance and the difference in absorption coefficient is equal to or less than the threshold value TH2 in any channel, the processor proceeds to step ST205.

In step ST205, the processor determines whether or not analysis of the absorption coefficients of both the first and second reference substances is completed. Herein, the analysis of the absorption coefficients of both the first and second reference substances has been completed. Therefore, the processor determines that output of an alert is not necessary, and ends step ST20. In this case, the process proceeds to step ST22, where examination of the subject is executed in accordance with the examination schedule for medical examination day q.

In the present embodiment, an alert was not output on medical examination day q. Therefore, after step ST20 ends, examination of the subject is executed. Similarly, the flow 200 is executed on medical examination day q and thereafter. Furthermore, if the difference in the absorption coefficient exceeds a threshold value, an alert is output, inspection of the X-ray generating device is executed, and an X-ray tube is repaired or replaced as necessary. Furthermore, when the CT system is restarted, the absorption coefficient of the calibration data obtained on the first day of restart is confirmed as the new reference absorption coefficient. Therefore, after the CT system is restarted, the change in the absorption coefficient over time is analyzed using the absorption coefficient obtained on the first day of restart as a reference value.

As described above, according to the present embodiment, absorption coefficient data is acquired every time a calibration scan is executed. Furthermore, the difference between the absorption coefficients is calculated to obtain an amount of change in the absorption coefficient over time, and whether the difference between the absorption coefficients is greater than a threshold value (TH1 or TH2) is determined. The threshold value TH1 or TH2 can be set to, for example, a value suitable for determining whether or not age-related deterioration has occurred in the X-ray generating device. Therefore, if age-related deterioration occurs in the X-ray generating device, an alert is output, and therefore, the user can know in advance that there is a possibility that the X-ray generating device is experiencing a malfunction or failure before the deterioration of the X-ray generating device appears as noise or artifacts in a CT image. This allows the user to take necessary measures, such as requesting a service for inspection or the like, before deterioration of the X-ray generating device appears as noise or artifacts in a CT image.

Note that in this example embodiment, for the sake of convenience, a single-row detector is adopted to describe a method for determining whether to output an alert, but the present invention can also be applied to a detector with a plurality of rows.

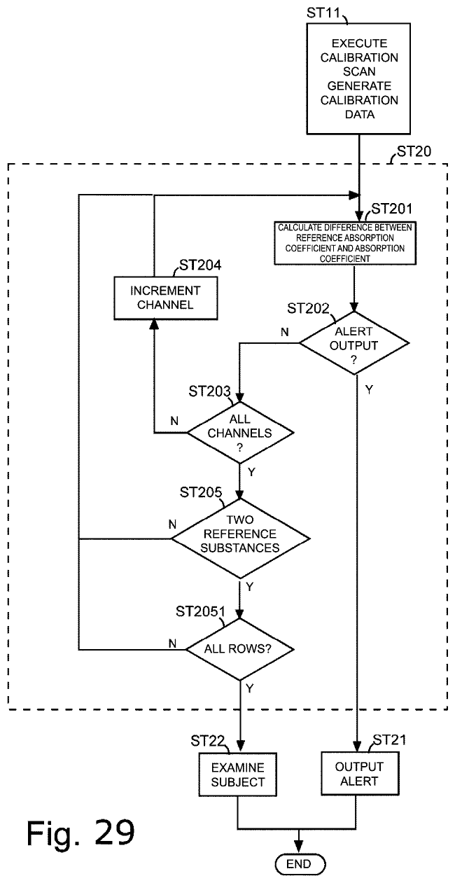

FIG. 29 is a diagram depicting an example of a flow of step ST20 when a detector with a plurality of rows is used. If compared, step ST20 depicted in FIG. 29 is different from step ST20 depicted in FIG. 9, in that step ST2051 is provided between step ST205 and step ST22.

In step ST2051, the processor determines whether or not the processes in steps ST201 to ST205 have been completed for all rows of the detector. If there is a row for which the processes in steps ST201 to ST205 have not been completed, the process returns to step ST1. On the other hand, if the processes in steps ST201 to ST205 have been completed for all rows of the detector, the process proceeds to step ST22.

By providing this step ST2051, it is possible to handle a detector with a plurality of rows. Note that in the present embodiment, the processes of steps ST201 to ST204 are executed in the order of channels 1 to n, but it is not necessary to execute the processes of steps ST201 to ST204 in the order of channels 1 to n, and the processes of steps ST201 to ST204 can be executed in an arbitrary channel order. Furthermore, when a detector with a plurality of rows is used, it is not necessary to execute the processes of steps ST201 to ST205 in the order of the first row to the m-th row, and steps ST201 to ST205 can be executed in an arbitrary row order. Furthermore, after the processes of steps ST201 and ST202 are executed for one or more channels in a certain row of the detector, the processes of steps ST201 and ST202 may be executed for one or more channels in another row of the detector.

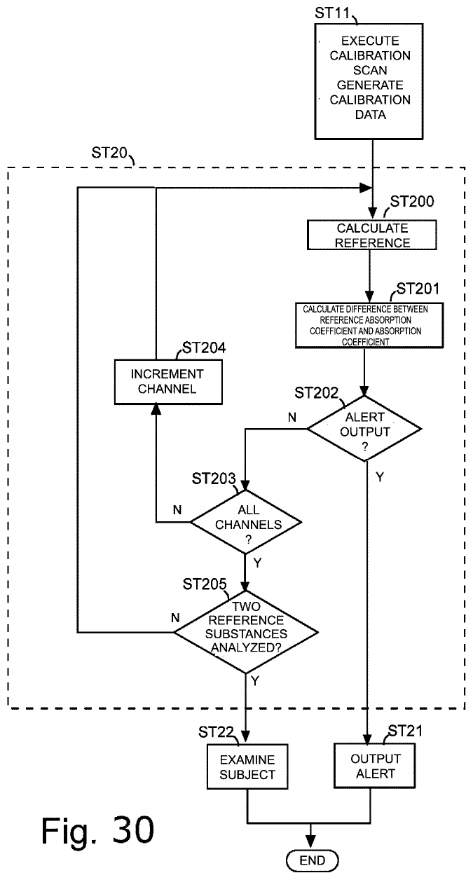

Furthermore, in the present embodiment, when the absorption coefficient data of the first reference substance is analyzed in step ST20, the absorption coefficients a₁₁ to a₁ₙ acquired on medical examination day 1 are used as the first reference absorption coefficients to determine whether to output an alert. Furthermore, when the absorption coefficient data of the second reference substance is analyzed in step ST20, the absorption coefficients b₁₁ to b₁ₙ acquired on medical examination day 1 are used as the second reference absorption coefficients to determine whether to output an alert. However, the reference absorption coefficient may be updated for each medical examination day (see FIG. 30).

FIG. 30 is an explanatory diagram of a flow of step ST20 when updating the reference absorption coefficient for each medical examination day.

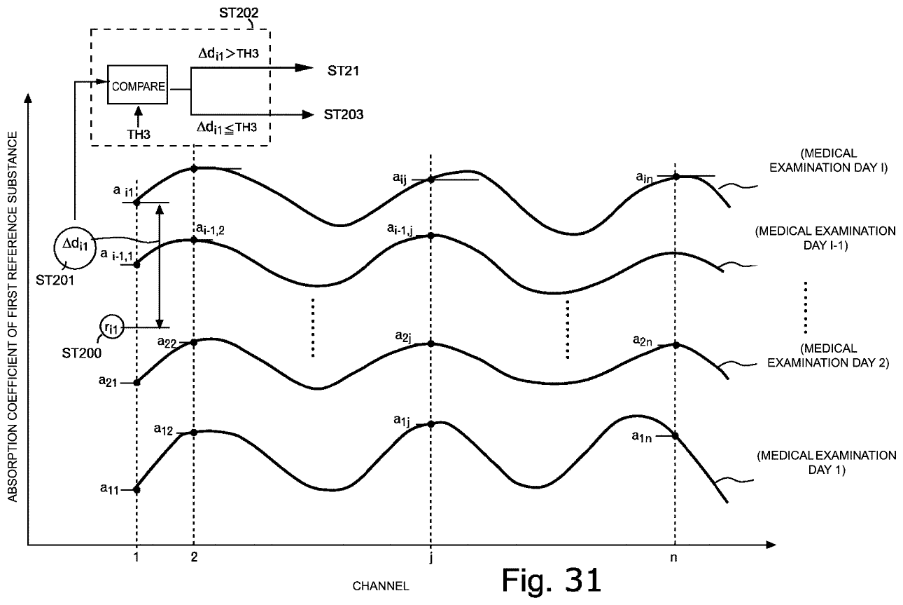

In FIG. 30, step ST20 includes step ST200 for calculating a reference absorption coefficient between steps ST11 and ST201. By providing step ST200, the reference absorption coefficient can be updated for each medical examination day and step ST20 can be executed. The process flow of step ST20 depicted in FIG. 30 will be described below with reference to FIG. 31.

FIG. 31 is a diagram depicting the absorption coefficient data g₁₁ to gᵢ₁ of the first reference substance acquired on medical examination days 1 to i. In step ST11, once the absorption coefficient data gᵢ₁ of the first reference substance on medical examination day i is acquired, the process proceeds to step ST20. In step ST20, whether or not to output an alert is determined based on change in the absorption coefficient over time. First, in step ST200, the processor calculates a reference absorption coefficient rᵢ₁ based on the absorption coefficients a₁₁ to a_{i-1,1} (absorption coefficients obtained by a previously executed calibration scan) of channel 1 of medical examination days 1 to i−1. The reference absorption coefficient rᵢ₁ is used as the first reference absorption coefficient in channel 1. The reference absorption coefficient rᵢ₁ may be, for example, an average value of the absorption coefficients a₁₁ to a_{i-1,1}, or a weighted average of the absorption coefficients a₁₁ to a_{i-1,1}. Once the reference absorption coefficient rᵢ₁ has been calculated, the process proceeds to step ST201.

In step ST201, the processor calculates the difference Δdᵢ₁ between a reference absorption coefficient r₁₁ and an absorption coefficient aᵢ₁ of medical examination day i. Once the absorption coefficient difference Δdᵢ₁ has been calculated, the process proceeds to step ST202.

In step ST202, the processor compares Δdᵢ₁ with a threshold value TH3, and determines whether or not to output an alert based on the comparison result thereof. For example, when Δdᵢ₁ exceeds the threshold value TH3 (Δdᵢ₁>TH3), the processor determines that the change in the absorption coefficient over time is large and therefore determines to output an alert. In this case, the process proceeds to step ST21, where the processor causes the display unit to output an alert.

On the other hand, if the difference Δdᵢ₁ in the absorption coefficients does not exceed the threshold value TH3 (Δdᵢ₁≤TH3), the change in the absorption coefficient over time is small, and therefore, the processor determines not to output an alert. In this case, the process proceeds to step ST203.

In step ST203, the processor determines whether or not steps ST200 to ST202 have been executed for all channels. Herein, steps ST200 to ST202 have been executed for channel 1, but have not yet been executed for the other channels 2 to n. Therefore, the process proceeds to step ST204, where the processor increments the channel from channel 1 to channel 2. Once the channel is incremented, the process returns to step ST200.

Similarly hereinafter, the loop of steps ST200 to ST204 is repeatedly executed. Therefore, the reference absorption coefficient is calculated for each channel, and whether or not to output an alert can be determined.

Note that although FIG. 31 describes that a first reference absorption coefficient, which is used when analyzing the change over time in the absorption coefficient of the first reference substance, is calculated, a similar method can also be used to calculate a second reference absorption coefficient, which is used when analyzing the change over time in the absorption coefficient of the second reference substance.

Furthermore, in FIG. 31, the reference absorption coefficient rᵢ₁ is calculated using all of the absorption coefficients a₁₁ to a_{i-1,1} of medical examination days 1 to i−1. However, it is not necessary to use all the absorption coefficients a₁₁ to a_{i-1,1} to calculate the reference absorption coefficient rᵢ₁, and the reference absorption coefficient rᵢ₁ may be calculated using at least one of the absorption coefficients a₁₁ to a_{i-1,1}.

Note that while FIG. 31 depicts a method for calculating the reference absorption coefficient rᵢ₁ on medical examination day i, a reference absorption coefficient for another medical examination day can also be calculated based on a past absorption coefficient in the same manner as for medical examination day i.

In another example embodiment, an example of calculating a representative value of an X-ray spectrum as an X-ray characteristic value will be described. Note that before describing the example embodiment in detail, features of the embodiment will be summarized as follows. In other words, in this example embodiment, a calibration scan is executed, and an X-ray spectrum corresponding to high kV and an X-ray spectrum corresponding to low kV are obtained based on data obtained by the calibration scan. Furthermore, the processor calculates a representative value of an X-ray spectrum corresponding to high kV and a representative value of an X-ray spectrum corresponding to low kV. The representative value of an X-ray spectrum is, for example, an average energy value or an integral energy value. The processor determines whether to output an alert based on change in the representative value of the X-ray spectrum over time. Therefore, by outputting an alert when there is a large change over time in the representative value of the X-ray spectrum, a user can be notified that there are signs of deterioration in the X-ray generating device before the image quality of a CT image deteriorates.

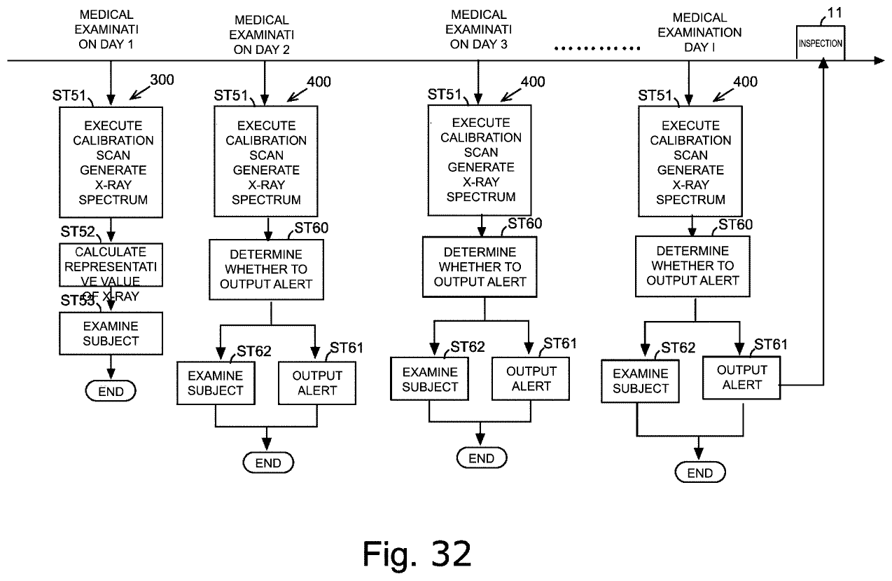

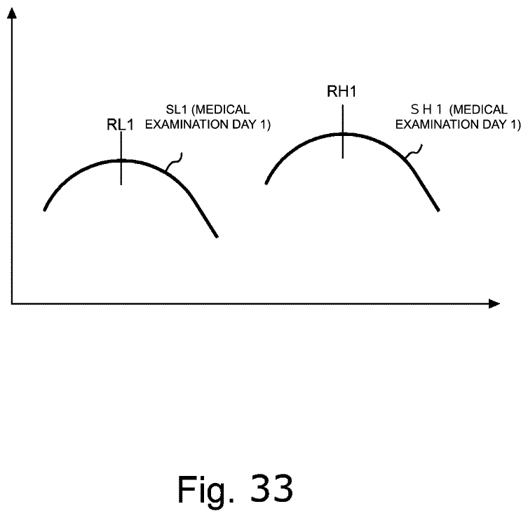

FIG. 32 is an explanatory diagram of a flow performed in an example embodiment. First, medical examination day 1 will be described. On medical examination day 1, a flow 300 is executed. In step ST51, a calibration scan is executed using kV switching in which the tube voltage applied to the X-ray tube 104A is alternately switched between a first tube voltage (low kV) and a second tube voltage (high kV). After the calibration scan is executed, the processor generates an X-ray spectrum based on data obtained in the calibration scan (see FIG. 33).

FIG. 33 is an explanatory diagram of the X-ray spectrum. An X-ray spectrum SL1 is an X-ray spectrum corresponding to low kV, and an X-ray spectrum SH1 is an X-ray spectrum corresponding to high kV. After the X-ray spectrums SL1 and SH1 are generated, the process proceeds to step ST52.

In step ST52, the processor calculates a representative value of the X-ray spectrum SL1. In the example embodiment, the processor calculates an average energy value RL1 of the X-ray spectrum SL1 as the representative value of the X-ray spectrum SL1. Furthermore, the processor calculates a representative value of the X-ray spectrum SH1. In the example embodiment, the processor calculates an average energy value RH1 of the X-ray spectrum SH1 as the representative value of the X-ray spectrum SH1. However, the representative value of the X-ray spectrum SL1 is not limited to the average energy value RL1, and an arbitrary value that represents a feature of the X-ray spectrum SL1, such as an integral energy value or the like, can be used as the representative value of the X-ray spectrum SL1. Similarly, the representative value of the X-ray spectrum SH1 is not limited to the average energy value RH1, and an arbitrary value that represents a feature of the X-ray spectrum SH1, such as an integral energy value or the like, can be used as the representative value of the X-ray spectrum SH1.

Note that the average energy value RL1 calculated on medical examination day 1 is stored as a first energy reference value serving as a criterion for determining whether or not to output an alert. Furthermore, the average energy value RH1 calculated on medical examination day 1 is stored as a second energy reference value serving as a criterion for determining whether or not to output an alert.

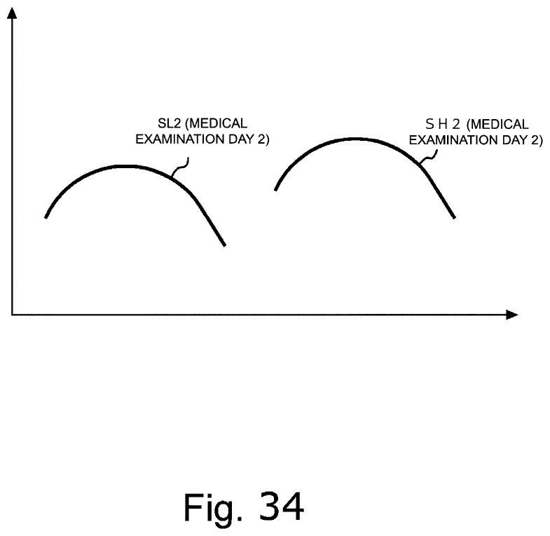

After step ST52 is executed, the process proceeds to step ST53, where examination of the subject is executed. Next, medical examination day 2 will be described. On medical examination day 2, a flow 400 is executed. In step ST51, a calibration scan is executed. After the calibration scan is executed, the processor generates an X-ray spectrum based on data obtained in the calibration scan. FIG. 34 is a diagram schematically depicting an X-ray spectrum SL2 corresponding to low kV generated on medical examination day 2, and an X-ray spectrum SH2 corresponding to high kV generated on medical examination day 2.

After step ST51 is executed, the process proceeds to step ST60. In step ST60, it is determined whether to output an alert based on change in the average energy value of the X-ray spectrum over time.

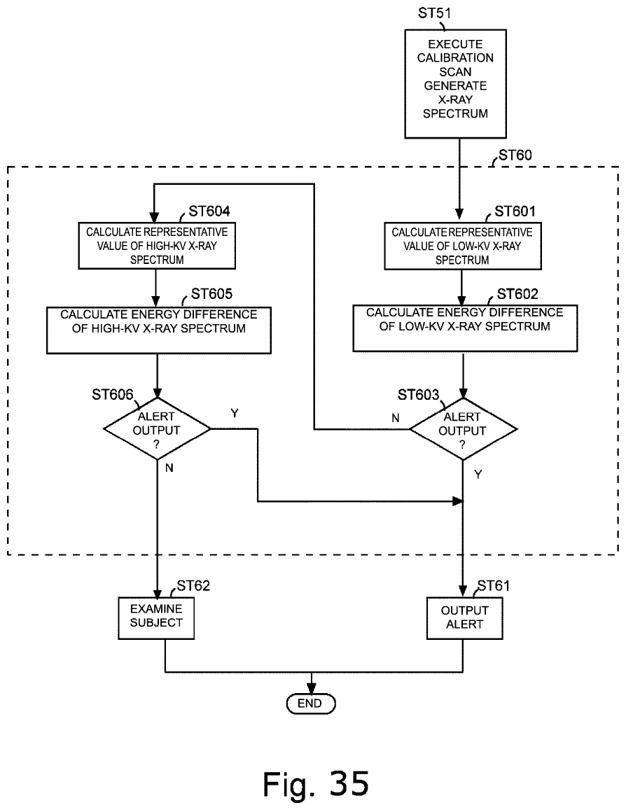

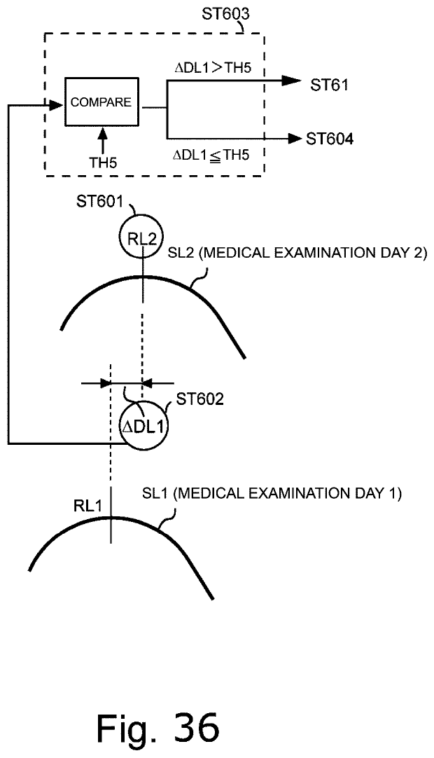

FIG. 35 is a diagram depicting an example of a flow of step ST60, and FIG. 36 is an explanatory diagram of the flow of FIG. 35. In step ST601, the processor calculates an average energy value RL2 of the X-ray spectrum SL2 on medical examination day 2. Once the average energy value RL2 has been calculated, the process proceeds to step ST602.

In step ST602, the processor calculates an energy difference ΔDL1 between the energy reference value RL1 and the average energy value RL2. After the energy difference ΔDL1 is calculated, the process proceeds to step ST603.

In step ST603, the energy difference ΔDL1 is compared with a threshold value TH5, and determines whether or not to output an alert based on the comparison result thereof. For example, if the energy difference ΔDL1 exceeds the threshold value TH5 (ΔDL1>TH5), the processor determines that the energy difference ΔDL1 is large and therefore determines to output an alert. In this case, the process proceeds to step ST61, where the processor causes the display unit to output an alert.

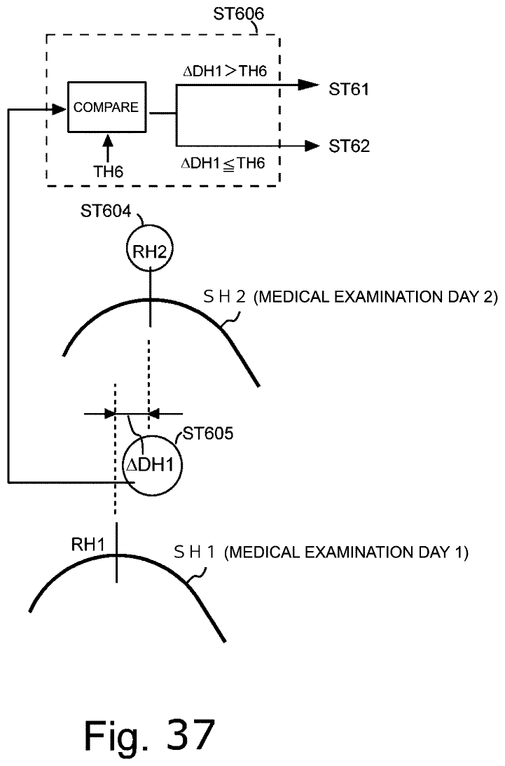

On the other hand, if the energy difference ΔDL1 does not exceed the threshold value TH5 (ΔDL1≤TH5), the energy difference ΔDL1 is small, and therefore, the processor determines not to output an alert. In this case, the process proceeds to step ST604 (see FIG. 37).

FIG. 37 is an explanatory diagram of steps ST604 to ST606. In step ST604, the processor calculates an average energy value RH2 of the X-ray spectrum SH2 on medical examination day 2. Once the average energy value RH2 has been calculated, the process proceeds to step ST605.

In step ST605, the processor calculates an energy difference ΔDH1 between the energy reference value RH1 and the average energy value RH2. After the energy difference ΔDH1 is calculated, the process proceeds to step ST606.

In step ST606, the energy difference ΔDH1 is compared with a threshold value TH6, and determines whether or not to output an alert based on the comparison result thereof. For example, if the energy difference ΔDH1 exceeds the threshold value TH6 (ΔDH1>TH6), the processor determines that the energy difference ΔDH1 is large and therefore determines to output an alert. In this case, the process proceeds to step ST61, where the processor causes the display unit to output an alert.

On the other hand, if the energy difference ΔDH1 does not exceed the threshold value TH6 (ΔDH1≤TH6), the energy difference ΔDL1 is small, and therefore, it is determined to not output an alert. In this case, both the change over time in the average low-kV energy value and the change over time in the average high-kV energy value were analyzed, but it was determined that an alert is not to be output. Therefore, the process proceeds to step ST62, and examination of the subject is executed in accordance with the examination schedule for medical examination day 2.

Similarly hereinafter, on medical examination days 3 to i, the flow 400 is also executed in the same manner as for medical examination day 2. Therefore, on medical examination days 3 to i, it is determined whether or not to output an alert based on the change over time in the average energy value of the X-ray spectrum by the same procedure as for medical examination day 2.

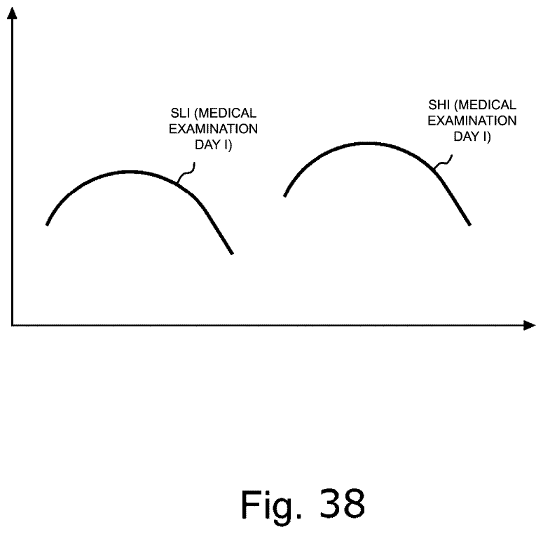

Next, medical examination day i will be described. In step ST51, a calibration scan is executed. After the calibration scan is executed, the processor generates an X-ray spectrum based on data obtained in the calibration scan. FIG. 38 schematically depicts the X-ray spectrum SLi corresponding to low kV generated on medical examination day i and the X-ray spectrum SHi corresponding to high kV generated on medical examination day i.

After step ST51 is executed, the process proceeds to step ST60. In step ST60, the processor determines whether to output an alert based on change in the average energy value of the X-ray spectrum over time.

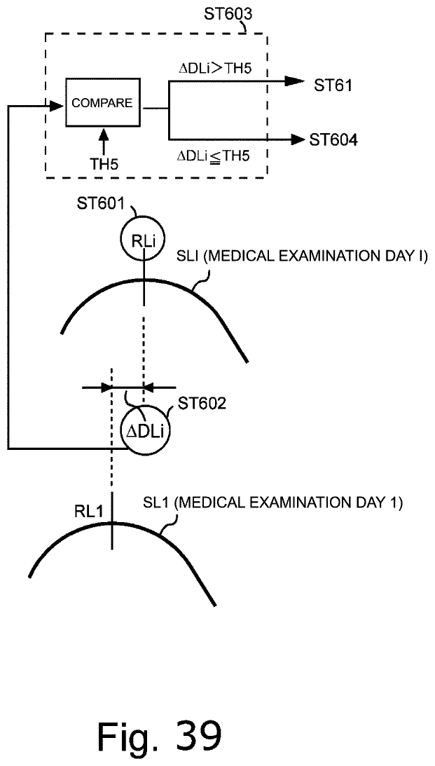

FIG. 39 is an explanatory diagram of step ST60. In step ST601, the processor calculates an average energy value RLi of the X-ray spectrum SLi on medical examination day i. Once the average energy value RLi has been calculated, the process proceeds to step ST602.

In step ST602, the processor calculates an energy difference ΔDLi between the energy reference value RLi and the average energy value RLi. After the energy difference ΔDLi is calculated, the process proceeds to step ST603.

In step ST603, the energy difference ΔDLi is compared with a threshold value TH5, and a determination is made as to whether or not to output an alert based on the comparison result thereof. For example, if the energy difference ΔDLi exceeds the threshold value TH5 (ΔDLi>TH5), the processor determines that the energy difference ΔDLi is large and therefore determines to output an alert. Herein, the energy difference ΔDLi satisfies ΔDLi>TH5. Therefore, an alert is output.

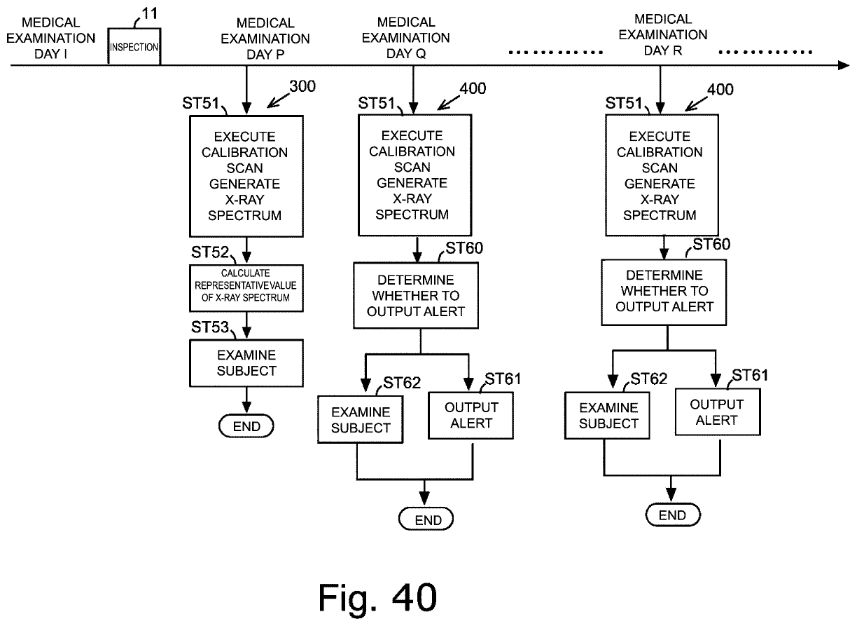

When the alert is output, the user can, for example, request a service to carry out an inspection 11 (see FIG. 2). As a result of the service inspection 11, if the X-ray generating device is acknowledged to have malfunctioned or failed, the X-ray generating device is repaired or replaced. After the X-ray generating device is repaired or replaced, an operation of the CT system is checked, and if the CT system is confirmed to be operating normally, the operation of the CT system is resumed. FIG. 40 is an explanatory diagram of a flow after the operation of the CT system is restarted. Herein, the inspection or the like is completed on medical examination day i, and operation of the CT system is resumed on a subsequent medical examination day p.

On medical examination day p, the same flow 300 as on medical examination day 1 is executed, and the average energy value of the X-ray spectrum is calculated. Furthermore, on medical examination day q and thereafter, the same flow 400 as for medical examination day 2 is executed. Therefore, the energy difference is calculated based on the average energy value calculated on the medical examination day p, and if the energy difference exceeds a threshold value, an alert is output. Therefore, on medical examination day q and thereafter, whether or not to output an alert can be determined based on the change over time in the average energy value of the X-ray spectrum.

As described above, according to the example embodiment, an X-ray spectrum is generated every time a calibration scan is executed. Furthermore, an energy difference is calculated to obtain the amount of change in the average energy value over time, and it is determined whether the energy difference is larger than a threshold value. Therefore, if the energy difference is large, an alert is output, and therefore, the user can know in advance that there is a possibility that the X-ray generating device is experiencing a malfunction or failure before the image quality of a CT image deteriorates. This allows the user to take necessary measures, such as requesting a service to inspect the X-ray generating device, or the like.

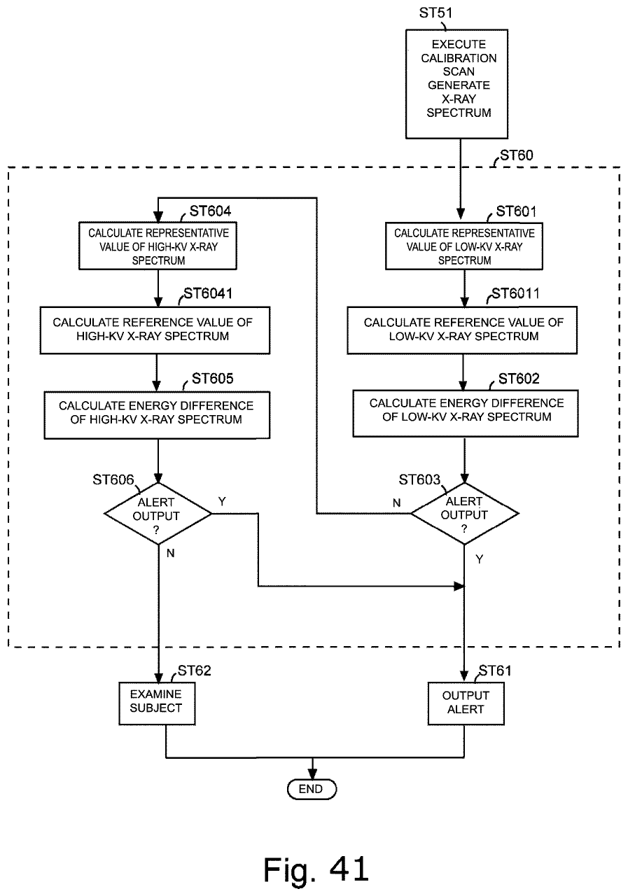

Note that in the example embodiment, the average energy value of the X-ray spectrum calculated on medical examination day 1 is determined as the energy reference value, and it is determined whether or not to output an alert. However, the energy reference value may be calculated for each medical examination day based on the average energy value of an X-ray spectrum acquired in the past (see FIG. 41).

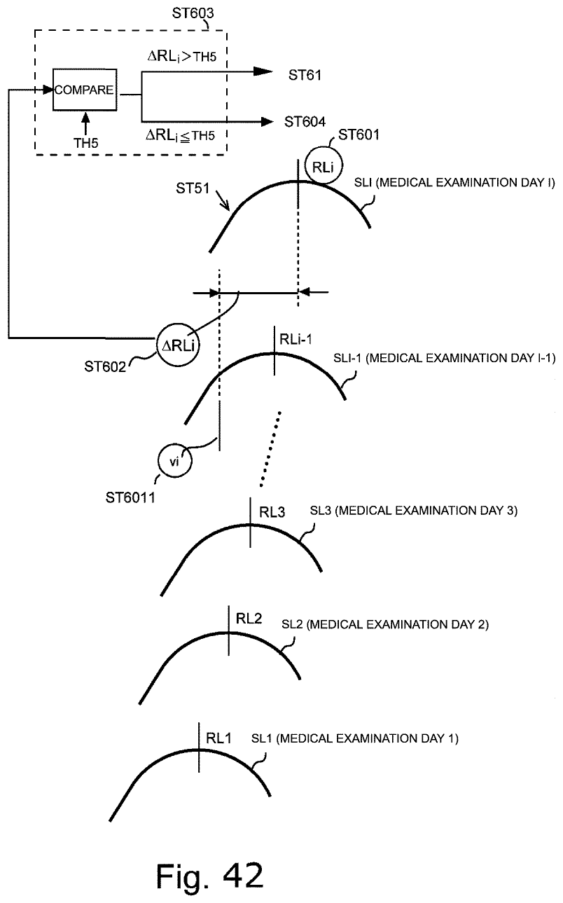

FIG. 41 is an explanatory diagram of a flow of step ST60 when calculating an energy reference value for each medical examination day. In FIG. 41, step ST60 includes step ST6011 between steps ST601 and ST602, in which an energy reference value of a low-kV X-ray spectrum is calculated. Furthermore, step ST60 includes step ST6041 between steps ST604 and ST605, in which an energy reference value of a high-kV X-ray spectrum is calculated. By providing steps ST6011 and ST6041, it is possible to calculate the energy reference value for each medical examination day and execute step ST60. The process flow of step ST60 depicted in FIG. 41 will be described below with reference to FIG. 42.

FIG. 42 is a diagram depicting the X-ray spectrums SL1 to SL corresponding to low kV acquired on medical examination days 1 to i. In step ST51, when the X-ray spectrum SLᵢ corresponding to low kV on the medical examination day i is acquired, the process proceeds to step ST601. In step ST601, the processor calculates the average energy value RLi of the X-ray spectrum SLi of medical examination day i. Once the average energy value RLi has been calculated, the process proceeds to step ST6011.

In step ST6011, the processor calculates a first energy reference value vᵢ based on average energy values RL1 to RLᵢ₋₁ of medical examination days 1 to i−1. The energy reference value vᵢ is used as a reference value for determining whether or not to output an alert on medical examination day i. The energy reference value vᵢ may be, for example, the average value of the average energy values RL1 to RLᵢ₋₁, or may be a weighted average of the average energy values RL1 to RLᵢ₋₁. Once the energy reference value vᵢ has been calculated, the process proceeds to step ST602.

In step ST602, the processor calculates an energy difference ΔRLi between the energy reference value vi and the average energy value RLi. After the energy difference ΔRLi is calculated, the process proceeds to step ST603.

In step ST603, the energy difference ΔRLi is compared with a threshold value TH5, and determines whether or not to output an alert based on the comparison result thereof. For example, if the energy difference ΔRLi exceeds the threshold value TH5 (ΔRLi>TH5), the processor determines that the energy difference ΔRLi is large and therefore determines to output an alert. In this case, the process proceeds to step ST61, where the processor causes the display unit to output an alert.

On the other hand, if the energy difference ΔRLi does not exceed the threshold value TH5 (ΔRLi≤TH5), the energy difference ΔRLi is small, and therefore, the processor determines not to output an alert. In this case, the process proceeds to step ST604.

Therefore, it is possible to determine whether to output an alert.

Note that although FIG. 42 describes that a first energy reference value, which is used when analyzing the change over time of the average energy value of an X-ray spectrum corresponding to low kV, is calculated, a similar method can also be used to calculate a second energy reference value, which is used when analyzing the change over time of the average energy value of an X-ray spectrum corresponding to high kV.

Furthermore, in FIG. 42, an energy reference value vi is calculated using all of the average energy values RL1 to RLᵢ₋₁ of medical examination days 1 to i−1. However, the energy reference value vi may be calculated using at least one of the average energy values RL1 to RLᵢ₋₁ of medical examination days 1 to i−1.

Note that although FIG. 42 depicts a method for calculating the energy reference value vi of medical examination day i, the reference energy value for another medical examination day can also be calculated based on the average energy value obtained from a past X-ray spectrum, in the same manner as for medical examination day i.

Note that in the present embodiment, an example is described in which the CT system is used as a medical system. However, the present invention is not limited to the CT system and can be applied to a system other than the CT system (e.g., PET-CT system), so long as the medical system irradiates an X-ray source onto the subject 112.

1. A medical system, comprising:
an X-ray generating device including an X-ray tube;
a detector for detecting X-rays irradiated from the X-ray generating device; and
one or more processors executing instructions to:
calculate a characteristic value of the X-rays irradiated from the X-ray generating device based on data on the X-rays detected by the detector, every time a prescribed calibration scan is executed; and
determine whether to output an alert based on change in the characteristic value over time.

2. The medical system according to claim 1, wherein the medical system periodically executes the prescribed calibration scan while alternately switching a tube voltage between a first tube voltage and a second tube voltage, and
the characteristic value includes an absorption coefficient of a first reference substance and an absorption coefficient of a second reference substance.

3. The medical system according to claim 2, wherein the one or more processors further execute instructions to calculate the absorption coefficient of the first reference substance and the absorption coefficient of the second reference substance every time the prescribed calibration scan is executed.

4. The medical system according to claim 3, wherein the one or more processors further executes instructions to:
calculate a first reference absorption coefficient serving as a criterion for determining whether to output an alert;
calculate a first difference between the first reference absorption coefficient and the absorption coefficient of the first reference substance; and
compare the first difference with a first threshold value and determining whether to output an alert based on a comparison result thereof.

5. The medical system according to claim 3, wherein the one or more processors further executes instructions to:
calculate a second reference absorption coefficient serving as a criterion for determining whether to output an alert;
calculate a second difference between the second reference absorption coefficient and the absorption coefficient of the second reference substance; and
compare the second difference with a second threshold value and determining whether to output an alert based on a comparison result thereof.

6. The medical system according to claim 5, wherein every time the prescribed calibration scan is executed, the one or more processors further executes instructions to:
calculate the first reference absorption coefficient based on the absorption coefficient of the first reference substance obtained by a previously executed prescribed calibration scan; or
calculate the second reference absorption coefficient based on the absorption coefficient of the second reference substance obtained by a previously executed prescribed calibration scan.

7. The medical system according to claim 6, wherein the first reference substance is water and the second reference substance is iodine or calcium.

8. The medical system according to claim 2, wherein when the alert is output, the medical system sends data indicating a time change in the absorption coefficient to a back office.

9. The medical system of claim 2, wherein the absorption coefficient of the first reference substance and the absorption coefficient of the second reference substance are calculated for each channel of each row of the detector.

10. The medical system according to claim 1, wherein the medical system periodically executes the prescribed calibration scan while alternately switching a tube voltage between a first tube voltage and a second tube voltage, and
the characteristic value includes a first representative value of a first X-ray spectrum corresponding to the first tube voltage and a second representative value of a second X-ray spectrum corresponding to the second tube voltage.

11. The medical system according to claim 10, wherein every time the prescribed calibration scan is executed, the one or plurality of processors calculates a first representative value of the first X-ray spectrum and a second representative value of the second X-ray spectrum.

12. The medical system according to claim 3, wherein the one or more processors further executes instructions to:
calculate a first energy reference value serving as a criterion for determining whether to output an alert;
calculate a first difference between the first energy reference value and the first representative value; and
compare the first difference with a first threshold value and determining whether to output an alert based on a comparison result thereof.

13. The medical system according to claim 3, wherein the one or more processors further executes instructions to:
calculate a second energy reference value serving as a criterion for determining whether to output an alert;
calculate a second difference between the second energy reference value and the second representative value; and
compare the second difference with a second threshold value and determining whether to output an alert based on a comparison result thereof.

14. The medical system according to claim 5, wherein every time the prescribed calibration scan is executed, the one or more processors further executes instructions to:
calculate the first difference based on a first representative value of the first X-ray spectrum obtained by a previously executed prescribed calibration scan; or
calculate the second difference based on a second representative value of the second X-ray spectrum obtained by a previously executed prescribed calibration scan.

15. The medical system according to claim 10, wherein the first representative value is an average energy value or an integral energy value of the first X-ray spectrum, and the second representative value is an average energy value or an integral energy value of the second X-ray spectrum.

16. The medical system according to claim 1, wherein the medical system executes a plurality of calibration scans, and one calibration scan of the plurality of calibration scans is executed as the prescribed calibration scan.
